(19) United States
(12) Patent Application Publication
(10) Pub. No.: US 2026/0259222 A1
(43) Pub. Date: Sep. 3, 2026

(54) METHOD OF DIAGNOSING WOUNDS

(71) Applicant: TRAUMA CARE CONSULT TRAUMATOLOGISCHE FORSCHUNG GEMEINNÜTZIGE GESELLSCHAFT MBH, Vienna (AT)

(72) Inventors: Mikolaj OGRODNIK, Vienna (AT); Nadja Anneliese Ruth RING, Vienna (AT); Helene DWORAK, Vienna (AT); Barbara SCHÄDL, Vienna (AT); Heinz REDL, Vienna (AT)

(21) Appl. No.: 18/878,650

(22) PCT Filed: Jun. 26, 2023
(86) PCT No.: PCT/EP2023/067248

§ 371 Date: Dec. 24, 2024

(30) Foreign Application Priority Data
Jun. 24, 2022 (EP) 22181050.0

(51) Int. Cl. G01N 33/68; G01N 33/58

(52) U.S. Cl. CPC G01N 33/6881; G01N 33/582; G01N 2440/14

(57) ABSTRACT

The present invention discloses a method comprising measuring an individual level or amount of presence, expression or activation of phosphorylated ribosomal protein S6 (p-rpS6) in a sample from a wound.

The present invention concerns diagnosing wounds and kits for use in such diagnosing methods.

The skin wounds of healthy patients normally heal without any problems. However, a large number of chronological and spatial changes in the cell composition of the skin are required in order to achieve complete healing of the tissue.

In general terms, wounding can be divided into three stages: i) the initial damage and its propagation, ii) the response of the organism and iii) tissue regeneration. The act of wounding causes damage of tissue and cells resulting in necrosis and apoptosis, often followed by an accumulation of debris and bleeding. This leads to rapid changes in ionic composition, oxygen concentration and redox status, breaching of the barrier layers and intrusion of microorganisms to the wounded area. The subsequent early responses of the organism include clotting, vasoconstriction, and mobilization of both immune and non-specialized cells to remove bacteria and debris. Soon after, the re-building of the damaged or missing tissue structures begins, involving synthesis of extracellular matrix (ECM), induction of cellular senescence, re-epithelialization and re-vascularization (Rodrigues et al., Physiol. Rev. 99 (2019):665-706). Owing to technical limitations, a great majority of the published research on the immediate reaction to wounding was conducted in indirect models of healing using in vitro 2D cell culture, in plants, invertebrates and fish, with only few studies done in mammals.

The process of wound healing can last up to two years and, in non-foetal tissue, is always associated with scar formation. This points to the enormous complexity of the wound healing process in the skin. In connection with wound healing, it is possible to distinguish the chronologically sequential, partially overlapping phases of coagulation, inflammation, proliferation and reorganization. During coagulation, blood platelets aggregate, with these platelets releasing growth factors and coagulation factors. A fibrin matrix is formed, with this matrix enabling cell migration to take place in the wound. An inflammatory reaction then occurs approx. 5-7 days after the injury. In connection with this, various cell types, in particular neutrophilic granulocytes and monocytes, migrate into the wound and release mediators of the inflammatory reaction. The proliferation phase serves the purpose of regenerating the damaged tissue and restructuring the tissue which has been regenerated. As already mentioned above, the processes involved include, in particular, neovascularization, fibroblast proliferation and re-epithelialization, brought about by the proliferation and differentiation of a very wide variety of skin cells and are regulated by a very wide variety of growth factors and/or combinations of these growth factors.

Since the above-listed growth factors appear to play a crucial role in achieving successful wound healing and an aesthetic result, the growth factors have in the past constituted an interesting subject for developing therapies. Since, however, the correct interplay of the numerous growth factors is influenced by a multiplicity of factors, such as the quantity, the spatial and chronological distribution, and the combination of growth factors, this approach turns out to be extremely complicated.

Sophisticated wound diagnostics are not currently available to the wound care professional. Wound diagnosis is empirical and heavily relies on the experience and knowledge of the practitioner. Improperly diagnosed wounds such as diabetic ulcers, venous-stasis wounds and pressure sores frequently result in negative, sometimes devastating outcomes. In the past, visual wound assessments provided an indication of the approximate depth, width and discharge status of a wound. For example, venous-stasis wounds continuously discharge fluid and tend to be wide and shallow (e.g. one to two centimeters deep). Diabetic ulcers tend to be drier and deeper relative to venous-stasis ulcers. Both types of wounds are frequently found on the lower extremities of patients. Diabetic ulcers are generally found on the undersides of the feet while venous-stasis ulcers are generally found on the calves and shins. However, these wounds may also be found on the upper torso and arms. Visual wound assessments have proven to be inadequate for proper diagnosis, especially for chronic wounds. Many patients who show signs of improvement, e.g. wound-size shrinkage and discomfort reduction, subsequently develop opportunistic infection including gangrene often requiring limb amputations to save their lives.

In most cases, aggressive therapeutic intervention is not indicated after initial visual wound assessment because the practitioner is unaware of the nature and actual extent of the wound. For example, the application of a debriding agent (enzymes which destroy necrotic tissue) would be inappropriate for a chronic or exacerbated wound. Compression bandages applied to a misdiagnosed pressure sore would compromise the healing of an actual venous stasis ulcer. Fundamentally, the practitioner does not know whether a wound is likely to heal or not. Therefore, several months of trial-and-error therapies face a patient with a chronic, nonhealing wound.

Moreover, it may be challenging within the course of wound diagnosis to state whether a 1) wound is vascularized (i.e. whether the lack of healing comes from the lack of vascularization or other causes) or whether 2) the wound is necrotic or where the necrotic region ends (i.e. how much should be cut).

The object of the present invention is therefore to find a novel marker which significantly improves diagnosis and/or prevention of wound healing disturbances, especially in clinical settings. Such a marker should be reliable and easy to be determined (e.g. by persisting long enough in a given patient to be appropriately detected, also in routine clinical practice specifically also in emergency hospitals).

Therefore, the present invention provides a method comprising the following steps:
measuring an individual level or amount of presence, expression or activation of phosphorylated ribosomal protein S6 (p-rpS6) in a sample from a wound; and
comparing the measured individual amount or level of p-rpS6 of the sample with a standard level or amount of p-rpS6 to assess tissue response to wounding.

The present invention further provides a method comprising the following steps:
measuring an individual level or amount of presence, expression or activation of phosphorylated ribosomal protein S6 (p-rpS6) in a sample from a wound; and
determining whether the measured individual amount or level of p-rpS6 of the sample is indicative for an impaired state of wound healing.

The present invention further provides a method comprising the following steps:
measuring an individual level or amount of presence, expression or activation of phosphorylated ribosomal protein S6 (p-rpS6) in a sample from a wound of a patient;
comparing the measured individual amount or level of p-rpS6 of the sample with a standard level or amount of p-rpS6 to assess tissue response to wounding; and
in case the comparison results in an assessment of impaired tissue response to wounding administering an effective amount of a therapeutic agent for improving wound healing in the patient.

The present invention further provides a method comprising the following steps:
measuring an individual level or amount of presence, expression or activation of phosphorylated ribosomal protein S6 (p-rpS6) in a sample from a wound; and
diagnosing an impaired wound healing risk if the measured individual amount or level of p-rpS6 of the sample is increased in comparison with the amount or level of a wound sample of a patient which is not affected by an impaired wound healing risk.

According to a specifically preferred aspect, the present invention provides a method comprising the following steps:
measuring the presence or absence of phosphorylated ribosomal protein S6 (p-rpS6) in a sample from a wound of a patient; and
identifying the absence of p-rpS6 in the sample as being indicative for a lack of vascularisation and/or lack of oxygenation of the wound and/or presence of necrotic tissue within the wound.

With the present invention it could be demonstrated that it is possible to visualize and measure response of the tissue to wounding as early as minutes and as long as weeks after skin damage, using a simple assay to detect presence or absence or stable modification of the protein rpS6, especially modification by phosphorylation. This new marker of wounding is activated by damage associated molecular patterns (DAMPs). p-rpS6 is therefore included by DAMPs, its initial induction depends on mTOR activity and it correlates (but does not depend on) with calcium influx. Its usage enables recognition of cells involved in skin response to damage, characterized by increased proliferation, altered rate of protein production, c-Fos expression and induction of cellular senescence markers. The present invention provides p-rpS6 as an early and stable marker to visualize skin response to wounding.

The present invention is based on the unexpected characteristic p-rpS6 has as a “wound marker”. In the course of the present invention, it was confirmed that p-rpS6 is a very early marker of wounding, independent of the nature of the wound. This means that in any wound, after a few minutes after injury which led to the wounding, p-rpS6 appears as a persistent wound marker and keeps its presence until the final healing of the wound. In contrast to other wound markers, which disappear after a certain onset period of the wound (but while the wound is still present) or which occur long after the wounding event (e.g. in the process of fibrinolysis and tissue regeneration), p-rpS6 accompanies the “normal” wound from its earliest stages to the final stage. This peculiarity makes p-rpS6 a viable and efficient marker for determining the presence of wounding and/or an appropriate wound healing process.

On the other hand, if in a wound no p-rpS6 is present, this is indicative of a severe problem which is directly connected to the wound healing process. For example, absence of p-rpS6 can be indicative for a lack of vascularisation and/or lack of oxygenation of the wound and/or presence of necrotic tissue within the wound.

Such necrotic tissue and processes may not be apparent by simple visual inspection on the wound. However, since necrotic tissue that is present in a wound presents a physical impediment to healing, it is crucial to remove necrotic tissue from wounds to improve the wound healing process, because wounds cannot heal when necrotic tissue is present. Necrotic tissue cannot be salvaged and must be removed to allow wound healing to take place. Necrotic tissue comprises a physical barrier that must be removed to allow new tissue to form and cover the wound bed but not always detectable by visual inspection. On the other hand, necrotic tissue is a vital medium for bacterial growth, and its removal also decreases wound bioburden.

With the present invention it is possible to clearly identify necrotic wound areas (by absence of p-rpS6) and to prevent overly excessive excision of wound areas which are in principle still viable (identified by the presence of p-rpS6). Therefore, the diagnostic according to the present invention allows wound treatment by surgical removal of necrotic tissue to be significantly more accurate to identify the tissue which is “truly necrotic” (characterised by the absence of p-rpS6).

As already explained, the present invention provides a very easy and reliable “yes” or “no” marker for wound diagnosis, because p-rpS6 is present in any “normal” wound and stays and persists until the end of the wound healing process. If, therefore, p-rpS6 is absent in a wound, this is indicative of a wound healing disturbance. On the other hand, p-rpS6 is otherwise not present in normal tissue (except at the top of the skin, in the stratum granulosum) so that no disturbances from other processes interfere with the methods according to the present invention.

The present invention provides a rapid and accurate method to diagnose wound status in a manner which permits meaningful assessment of the wound for proper treatment and for assessing the advisability of tissue (e.g. skin) grafts. As uniquely determined by the present invention, chronic or exacerbated wounds can now be diagnosed by measuring p-rpS6 presence, amounts and/or levels, especially the mere presence or absence of p-rpS6, in wound samples. In accordance with the present invention, the lack or absence of p-rpS6 in a wound sample relative to the actual and persistent presence levels in normal wounds without a risk of developing impaired wound healing correlates with a lack of vascularisation and/or lack of oxygenation of the wound and/or presence of necrotic tissue within the wound.

Wound therapy assessment is therefore greatly enhanced by the present invention. In the past, the practitioner would treat a wound simply based on its outward appearance. For example, a venous-stasis wound was conventionally treated with antibiotics, alginate dressings and/or tissue grafts. The process was hit or miss; if the prescribed treatments and/or tissue grafts were successful, then the patient was helped in the short term. But if the treatments were inappropriate, the patient could be faced with months of useless medication or additional tissue grafts, at significant cost. With the present invention, the practitioner can now quickly, more accurately and non-invasively determine the nature and severity of a wound, especially with respect to necrotic areas or areas with lack of oxygenation or vascularisation, and prescribe an appropriate therapy for short-term remediation. The present invention provides the practitioner with crucial information about the nature and extent of the wound and whether problems concerning e.g. necrotic tissue are present or likely to appear or not. Aggressive wound therapy can now be implemented or avoided depending on the p-rpS6 levels of a wound sample of a patient as determined by the present invention.

Within the meaning of the present invention, “wound healing” is to be understood as meaning the process of healing an injury to the skin, such as incision wounds, abrasion wounds, burn wounds, or the chafing of the skin, for example as the result of continuous stress, for example decubitus or necrotic processes, for example necrobiosis lipoidica.

rpS6 is an evolutionary conserved protein that spans 236 to 253 residues in species as remote as yeast, plants, invertebrates, and vertebrates, yet no homology with any ribosomal protein in Escherichia coli or archaebacteria has been detected. The role of rpS6 was first addressed by conditional knockout of the respective gene in adult mouse liver. In this connection it was shown that hepatocytes that lacked rpS6 gene failed to synthesize the 40S ribosomal subunit and consequently to proliferate following partial hepatectomy. This failure to progress through the cell cycle correlated with a block in expression of cyclin E gene. Nonetheless, the expression of rpS6 gene was not required for liver growth when starved mice were refed. Moreover, the relative engagement of liver ribosomes in translation, as exemplified by their polysomal association, was indistinguishable between rpS6-containing and lacking hypertrophying livers. The critical role of rpS6 is not confined to the regenerating liver, as thymus-specific knockout of both rpS6 alleles, but not conditional deletion of one allele, had devastating effect on the gland development. rpS6 heterozygosity (rpS6 wt/del), however, had a remarkable effect on the number of mature T cells in peripheral lymphoid organs (spleen and lymph nodes). rpS6 was described already in the 1970ies as the only ribosomal protein that undergoes phosphorylation in rabbit reticulocytes during rat liver regeneration. The phosphorylation sites in rpS6 in mammals and Xenopus laevis have been mapped to five clustered residues, S235, S236, S240, S244, and S247, whose location at the carboxy terminus of higher eukaryotes is evolutionarily conserved. It has been proposed that phosphorylation progresses in an ordered fashion, with Ser236 as the primary phosphorylation site. Numerous reports have demonstrated that rpS6 is subject to phosphorylation in response to multiple physiological, pathological, and pharmacological stimuli. Notably, this modification can be detected in both the cytosol and the nucleus. However, distinct nuclear/cytoplasmic distribution of rpS6 phosphorylated at different sits has been noticed for primary human cells, yet the physiological significance of this compart-mental preference is not clear (Meyuhas, Int. Rev. Cell Mol. Biol. 320 (2015):41-73).

Phosphorylation of rpS6 is mainly performed by the S6 Kinase (S6K). Mammalian cells contain two forms of S6K, S6K1 and S6K2 (also known as S6Ka and S6Kb respectively), which are encoded by two different genes and share a very high level of overall sequence homology. S6K1 has cytosolic and nuclear isoforms (p70 S6K1 and p85 S6K1, respectively), whereas both S6K2 isoforms (p54 S6K2 and p56 S6K2) are primarily nuclear and partly associated with the centrosome. Phosphorylation of rpS6 at Ser235 and Ser236 (Ser235/236) can still be detected, albeit at a much lower level, in cells lacking both S6K1 and S6K2. It could be shown that this phosphorylation is carried out, both in vitro and in vivo, by 90-kDa rpS6 Kinase (RSK; four RSK genes (RSK1 to RSK4) have been identified in mammals), which phosphorylates rpS6 exclusively at Ser235/236 in a response to serum, growth factors, tumor-promoting phorbol esters, and oncogenic Ras. The consensus recognition sequences of S6K and RSK are similar. Notably, however, the sequence context of serine 236 in rpS6 is the only one, among the phosphorylatable serine residues, that conforms to the consensus recognition sequence of these enzymes. Protein kinase A (PKA) was also shown to phosphorylate rpS6 in pancreatic cells, fibroblasts, pheochromocytoma, neuroblastoma, and kidney cells. This phosphorylation was reported to be limited to Ser235/236. Also Casein Kinase 1 (CK1) has been shown to phosphorylate rpS6 (selectively at Ser247) (Meyuhas, Int. Rev. Cell Mol. Biol. 320 (2015):41-73).

Chen et al reported that hyperphosphorylation of rpS6 predicts unfavourable clinical survival in non-small cell lung cancer (J. Exp. Clin. Cancer Res. 34 (2015), 34). WO 2007/090032 A2 discloses rpS6 as a pharmacodynamic marker for HSP90 inhibition, especially in connection with tumor samples.

The steady state level of rpS6 phosphorylation is the product of a dynamic equilibrium between the activities of the respective kinases and the opposing phosphatases. Nonetheless, the fluctuations in rpS6 phosphorylation have been attributed, in nearly all the relevant reports, to parallel changes in the kinase(s) activity. In a few cases, however, the phosphorylation status of rpS6 has been primarily ascribed to the activity of a phosphatase rather than a kinase. Current data indicate that protein phosphatase type 1 (PP1) is the primary phosphatase of rpS6 (Meyuhas, Int. Rev. Cell Mol. Biol. 320 (2015):41-73).

Since substitution of the two phosphorylatable serine residues to alanines in yeast rpS6 had no detectable effect on yeast growth under a wide variety of nutritional conditions. It was concluded that rpS6 phosphorylation has no obvious role in protein synthesis or other cellular functions in yeast. It appears therefore, that protein synthesis, at least in this cell type, is downregulated by rpS6 phosphorylation. On the other hand, S6K has also been implicated as an important player in the development of cancer and SK& and the phosphorylation state of Ser235/236 in rpS6 were therefore used as a biomarker for activation of the PI3K/mTORC1/S6K pathway in tissue samples from tumor biopsies or transplants (WO 2017/040960 A1). However, rpS6 or its phosphorylation have not yet been described in connection with wounds. In November 2022, one of the inventors of the present invention provided the presentation “The p-rpS6-zone Delineates Wounding Responses and the Healing Process”. This presentation was announced before the actual presentation as “The Zone of Tissue Activation Delineates Immediate and Long-Term Response of Skin to Wounding and Associates with Markers of Senescence and Regeneration”. The actual title of the presentation was not published before the actual presentation in November 2022.

According to a preferred embodiment of the present invention, absence of p-rpS6 in a wound sample is indicative for a risk for developing a chronic wound, including venous, pressure and diabetic ulcer; a hypertrophic scarring, a hypoxia-injured tissue, an impaired re-epithelialization, or an increased cell death in said tissue (Hakkarainen et al., Curr. Probl. Surg. 51 (2014), 344-362):

Necrotizing soft tissue infections (NSTIs) rank among the more difficult disease processes encountered by physicians and surgeons. NSTIs can arise primarily in the dermis and epidermis, but they more commonly affect the deeper layers of adipose tissue, fascia, or muscle. NSTIs are typically caused by toxin-producing bacteria and are characterized clinically by very rapid progression of disease with significant local tissue destruction. Varying amounts of early or late systemic toxicity depend on the strain of bacteria and toxins produced. Once symptomatic, the progression of disease is typically measured in hours; early diagnosis and treatment are crucial to survival. Most commonly, NSTIs are described as 1 of 3 bacteriologic classes: Type I NSTIs (classically polymicrobial infections with various species of gram-positive cocci, gram-negative rods, and anaerobes, including clostridial species), Type II NSTIs (infections involve group A β-hemolytic streptococci (GAS), either alone or in combination with staphylococcal species), and Type III NSTIs (caused by gram-negative marine organisms, most commonly V. vulnificus). There have also been multiple scoring systems developed to facilitate the diagnosis of NSTI and to establish risk thresholds; however, the gold standard for diagnosis of NSTI before the present invention still remains operative exploration in the setting of high clinical suspicion (Hakkarainen et al., 2014).

According to a specifically preferred aspect, the present invention also relates to a method for identifying necrotic tissue within a wound comprising the following steps:
measuring the presence or absence of phosphorylated ribosomal protein S6 (p-rpS6) in a sample from a wound; and;
identifying the area from which the sample of the wound was derived as being necrotic tissue; and, if applicable,
removing the necrotic tissue from the wound.

According to a preferred embodiment of the present invention, the method is a method of determining a diagnosis of the wound in a subject.

Preferably, the p-rpS6 measurement in the method according to the present invention is repeated for at least 1, 2, 3, 4, or 5 times.

The method according to the present invention may preferably be repeated during a course of monitoring the wound.

The present method is also suitable to computerization. Specifically, the determination or comparison is therefore preferably implemented and/or performed at least in part by a computer, by a computer processor and/or by a computer program. In addition, the present method therefore preferably comprises communicating a result via a communication medium, preferably wherein the communication medium is a computer file, an email, a fax, or a paper document.

In addition to the method according to the present invention, it is always advantageous to additionally evaluate the wound visually or to apply further wound diagnosis methods, such as quantitating cortisol in wound fluids (EP 0 902 288 A2).

The wound sample used in the method according to the present invention is preferably a tissue sample from the wound, preferably from a marginal edge of the wound or a cartilage sample from the wound or from the vicinity of the wound, especially a sample comprising of keratinocytes or other types of cells isolated or purified from the wound. A “sample of a wound” or “wound sample” is therefore preferably any sample taken from a wound or its vicinity (e.g. until approximately about 2 mm from the wound; this can—for small wounds—also be less). Usually, the wound sample is taken as a biopsy at the edge of the wound (e.g. from a “marginal edge” in order to disturb the wound healing process to the smallest extent possible/feasible). On the other hand, there is also the possibility of leakage these (soluble) wound markers into fluid samples collected from the wound (e.g. a body fluid sample (such as blood) taken from the wound) or other body fluids, e.g. after entrance of these factors into those body fluids or into circulating cells which ingested part of wound tissue e.g. macrophages. Accordingly, a “sample from a wound” according to the present invention may—in addition to a sample taken from the wound or from the vicinity of the wound—also be taken from the blood circulation of a patient with a wound to detect p-rpS6, its derivative or a related molecule leaked from the wound into blood circulation of being ingested at the wound site by cells then circulating then further in the blood circulation of the patient with the wound. For example, the sample from a wound may also be a body fluid sample and/or tissue sample and/or cell sample from the circulation of the patient having the wound.

Preferably, the measured levels and amounts of p-rpS6 are normalized fluorescence intensity (NFI) per mg protein, especially wherein the measured values are normalized expression values.

The method according to the present invention may be used for diagnosing any wounds (specifically under consideration of the teaching to the various wounds provided herein). Preferably, the wound is a burn injury, an excision or incision injury, a puncture injury, a diabetic ulcer, a pressure ulcer, a venous stasis ulcer, a radiation ulcer, a skin injury, an unhealed surgical wound, a wound from a surgical procedure, a wound from a peripheral vascular disease, a wound from a complication of trauma, a wound in a cancer patient, a wound in a patient receiving a steroid therapy, a wound from an inflammatory skin disease, a chronically impaired cutaneous wound, or any combination thereof, especially wherein the wound is a burn injury, an excision injury, or a puncture injury.

According to another aspect, the present invention also relates to a method of treating an individual having a wound with an individual level or amount of presence, expression or activation of p-rpS6 in a sample from said wound indicating an impaired tissue response to wounding by administering an effective amount of a therapeutic agent for improving wound healing in the patient.

The present method preferably applies an established treatment regimen for difficult wounds, if diagnosed by the present invention to exhibit a risk for impaired wound treatment. Established treatments comprise administering to the individual a pharmaceutical composition that comprises an inhibitor of POKinase signaling, preferably selected from an inhibitor of POKinase complex, Akt, mTOR, 4E.BP1, Ribosomal Protein S6 Kinase, HIF-Iα, PTEN, POKinase, IGFRPB3, Src, GSK3, β-catenin, any protein that is a member of the insulin signaling or POKinase signaling pathways of proteins; an activator of the thrombin receptor, preferably thrombin or a thrombin receptor activating peptide (TRAP), such as TRAP6, TRAP7 or TRAP8; an activator of insulin signaling through IGF-1R, preferably selected from the group consisting of insulin, insulin-like growth factor 1 (IGF-1), IGF-2, PTEN, POKinase, mTOR, 4E.BP1, Ribosomal Protein S6 Kinase, HIF-1a, PTEN, IGFRPB3, Src, GSK3, β-catenin, any protein that is a member of the insulin signaling or POKinase signaling pathways of proteins; or any combination thereof (WO 2016/205259 A1).

According to a further aspect, the present invention also relates to a kit, preferably for use in the method according to the present invention, comprising
means for taking a wound sample from a patient; and
means for detecting the level or amount of presence, expression or activation of p-rpS6.

Preferably, the wound sample is a tissue sample of body fluid sample of the patient having the wound, such as a wound tissue sample or a blood, serum or plasma sample (or a sample comprising a mixture of tissue (or cells) and body fluid, such as a wound biopsy or a blood sample from the wound wherein optionally cellular components have been removed before measuring). As already stated above, it is preferred to take the sample directly from the wound but taking the sample from the circulation is also possible, preferably if this sample from the circulation is taken in addition to the sample taken directly from the wound.

Preferably, the means for detecting the level or amount of presence, expression or activation of p-rpS6 are selected from labelled specific p-rpS6-binding molecules, preferably fluorescence-labelled anti-p-rpS6-antibodies, especially fluorescence-labelled monoclonal anti-p-rpS6-antibodies.

According to a further aspect, the present invention relates to a method for visualizing hair growth or functionality of hair follicles (as disclosed in e.g. Ito et al., Nature 447 (2007), 316-320) in a sample comprising hair follicles, comprising the following steps:
measuring an individual level or amount of presence, expression or activation of phosphorylated ribosomal protein S6 (p-rpS6) in a sample comprising hair follicles; and
comparing the measured individual amount or level of p-rpS6 of the sample with a standard level of healthy hair follicles or amount of p-rpS6 to assess the functionality of the hair follicle;
or
a method comprising the following steps:
measuring an individual level or amount of presence, expression or activation of phosphorylated ribosomal protein S6 (p-rpS6) in a sample comprising hair follicles; and
determining whether the measured individual amount or level of p-rpS6 of the sample is indicative for an impaired state of the hair follicle;
or
a method comprising the following steps:
measuring an individual level or amount of presence, expression or activation of phosphorylated ribosomal protein S6 (p-rpS6) in a sample comprising hair follicles of a patient;
comparing the measured individual amount or level of p-rpS6 of the sample with a standard level or amount of p-rpS6 to assess healthy hair growth; and
in case the comparison results in an assessment of impaired hair follicle function administering an effective amount of a therapeutic agent for improving hair growth in the patient.

The present invention also relates to a method comprising the following steps:
measuring the presence or absence of phosphorylated ribosomal protein S6 (p-rpS6) in a sample comprising hair follicles of a patient; and
identifying the absence of p-rpS6 in the sample as being indicative for an impaired hair growth.

The present invention also relates to a method comprising the following steps:
measuring an individual level or amount of presence, expression or activation of phosphorylated ribosomal protein S6 (p-rpS6) in a sample comprising hair follicles; and
diagnosing an impaired hair follicle activity if the measured individual amount or level of p-rpS6 of the sample is increased in comparison with the amount or level of a sample of a patient which is not affected by an impaired hair growth.

Preferably, the present methods may be combined with other methods for determining the activity and functionality of hair follicles. Accordingly, the present method comprises that the hair follicles are also tested with respect to Lef1 and/or p-STAT3 Y705, wherein presence of Lef1 and/or STAT3, especially p-STAT3 Y705, indicates appropriate function of the hair follicles and healthy hair growth (Ito et al., Nature 447 (2007), 316-320; Miyauchi et al., Front Immunol. 12 (2021), 663177). The markers Lef1 and pSTAT3 therefore represent “active” hair follicles, i.e. those that are growing hair. As p-rpS6 co-localized with them (i.e. if it is detected to be in the same place) shows that p-rpS6 is a marker of hair growth and thus its lack represents the lack of hair growth.

According to a preferred embodiment, the kit further complies an instruction manual.

The present invention is further illustrated by the following examples and the figures, yet without being limited thereto.

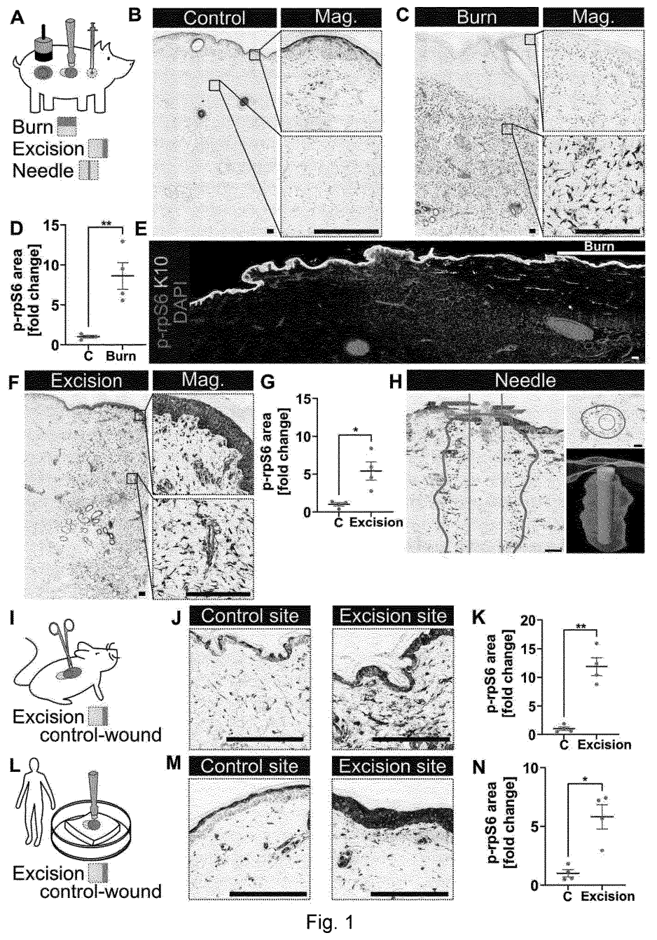

FIG. 1 shows that RpS6 phosphorylation marks a zone of tissue activation in response to wounding, a damage response which is conserved in mammals and between types of injury
(A) Experimental model. Pigs were injured by 60° C. burn, excision wound or needle prick and samples were collected 1.5 h later.
(B) Images of porcine skin biopsies representing p-rpS6 staining in control skin. Micrographs show regions of epidermis and dermis.
(C) Images of porcine skin biopsies representing p-rpS6 staining in 60° C. burn injury. Micrographs show regions of epidermis and dermis.
(D) Quantification of the area of p-rpS6 in colour-deconvolved images of control and burnt skin.
(E) An image of porcine skin biopsy collected from an edge of a 60° C. burn injury representing p-rpS6 (red) and K10 (green) fluorescent immunostainings. Nuclei are stained with DAPI (blue). The area that was burned is marked top right with a white bar and a label “Burn”.
(F) Images of porcine skin biopsies representing p-rpS6 staining in excision wound. Micrographs show regions of epidermis and dermis.
(G) Quantification of the area of p-rpS6 in colour-deconvolved images of control and skin wounded by excision injury.
(H) Images of porcine skin biopsies representing p-rpS6 staining in needle prick. For this experiment, the sample was sectioned into 40 slices 4 μm-thick with 20 μm gaps between each slice. The samples were stained for p-rpS6 and the lateral view (left panel) of the damaged area and p-rpS6-zone was assembled from individual slices (representative one in the top right panel). These 40 slices were used to create a 3D projection representing regions of tissue positive for p-rpS6 staining (bottom right panel). The green zone shows the needle injection damage and the red zone represents cells positive for p-rpS6.
(I) Experimental model. Mice underwent surgical removal of skin (1 cm diameter, full-thickness excision) and were sacrificed 0.5 1.5 h later.
(J) Images of mouse skin showing p-rpS6 staining. Micrographs show p-rpS6 staining in regions proximal (Excision site) and distal (Control site) to the wound site (the right side of the image).
(K) Quantification of the area of p-rpS6 in colour-deconvolved images of the control site and excision site of the excision wound in mouse skin.
(L) Experimental model. Skin was collected as a by-product of liposuction. After adjusting to ex vivo conditions skin was injured by inflicting an excision wound using a biopsy punch. After 1.5 h tissue was collected from the edge of the wound.
(M) Images of human ex vivo skin showing p-rpS6 staining. Micrographs show p-rpS6 staining in regions proximal (excision site) and distal (control site) to the wound site (the right side of the image).
(N) Quantification of the area of p-rpS6 in colour-deconvolved images of the control site and excision site of the excision wound in human skin ex vivo.

Data are from n=4 pigs per group for (D) and (G); n=4 mice per group for (K); n=4 human subjects for (N). Mean±SEM plotted. For all graphs unpaired t-test was used. *p<0.05, **p<0.01. The scale bars for all the images are 100 μm.

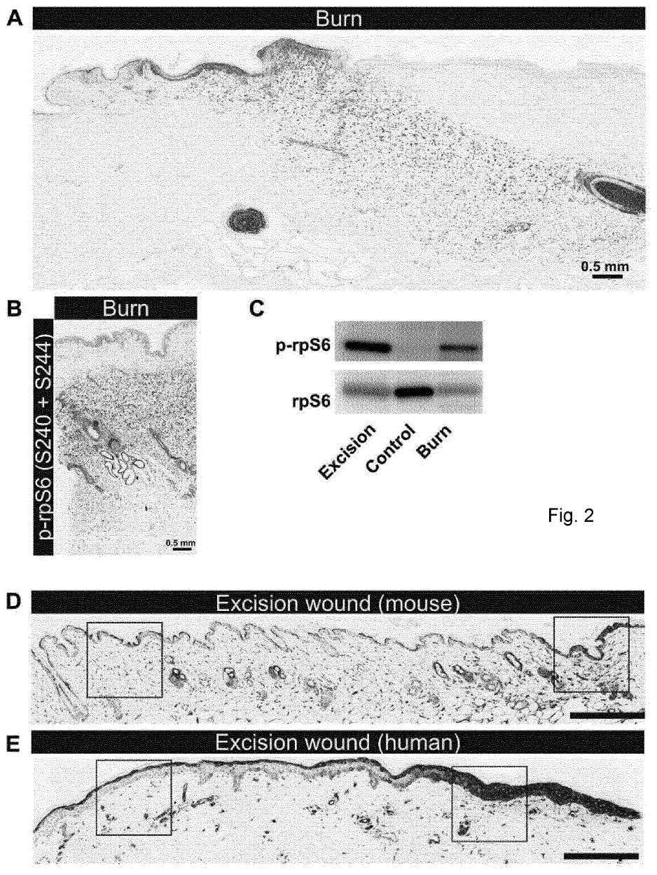

FIG. 2 shows that RpS6 phosphorylation marks a zone of tissue activation in response to wounding, a damage response which is conserved in mammals
(A) An image of porcine skin biopsy collected from a side of a 60° C. burn injury representing p-rpS6 IHC staining.
(B) Image of porcine skin biopsies representing the alternative phosphorylation site of p-rpS6 (5240/5244) in 60° C. burn injury sample.
(C) Western blot results for quantity of p-rpS6 and GAPDH (loading control) in porcine skin samples collected 1.5 h after excision injury, 60° C. burn injury or control.
(D) An image of mouse skin showing p-rpS6 staining. Frames mark regions for which micrographs were taken (for the FIG. 1) to show p-rpS6 staining in regions proximal (Excision site) and distal (Control site) to the wound site (the right side of the image).
(E) An image of human ex vivo skin showing p-rpS6 staining. Frames mark regions for which micrographs were taken (for the FIG. 1) to show p-rpS6 staining in regions proximal (excision site) and distal (control site) to the wound site (the right side of the image).

The scale bars for (A) and (B) show 500 μm and for (D) and (E) show 100 μm.

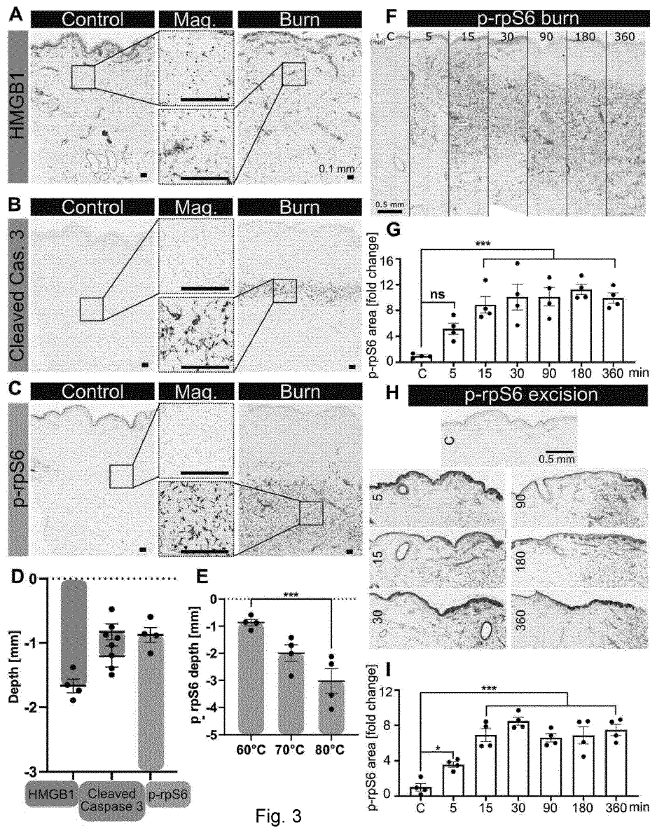

FIG. 3 shows that the induction of the p-rpS6-zone is an early-onset and long-term response that stratifies cell death and survival responses
(A) HMGB1 protein is present in the nuclei of undamaged skin cells and leaks to cytoplasm in burn injury. Images of porcine skin biopsies representing HMGB1 staining in control and 60° C. burn injury. Micrographs show magnified regions of dermis.
(B) Cleaved caspase 3 is absent in control skin but present in burn injury recognizable as a zone of apoptosis. Images of porcine skin biopsies representing cleaved caspase 3 staining in control and 60° C. burn injury. Micrographs show magnified regions of dermis.
(C) Images of porcine skin biopsies representing p-rpS6 staining in control and 60° C. burn injury. Micrographs show magnified regions of dermis.
(D) Quantification of the depth and thickness of the responses to the 60° C. burn injury 1.5 h from the time of the burn. Purple marks the thickness of layer of HMGB1 leakage, red shows the depth of initiation (top datapoints) and thickness (bottom datapoints) of the cleaved caspase 3 layer, while green shows the depth of initiation of the p-rpS6 layer.
(E) Quantification of the depth of initiation of the p-rpS6-zone in response to the 60, 70 and 80° C. burn injury at 1.5 h after the burn.
(F) Representative images of immunohistochemical staining against p-rpS6 in control conditions and 5 min, 15 min, 30 min, 1.5 h, 3 h and 6 h after 60° C. burn injury.
(G) Quantification of the colour-deconvolved images stained for p-rpS6 in control sample and at set timepoints after induction of burn injury.
(H) Representative images of immunohistochemical staining against p-rpS6 in control conditions and 5 min, 15 min, 30 min, 1.5 h, 3 h and 6 h after induction of excision wound. Wound is on the right side.
(I) Quantification of the colour-deconvolved images stained for p-rpS6 in control sample and at set timepoints after induction of excision wound.

Data are from n=4 pigs per group. Mean±SEM plotted. For (E), (G) and (I) one-way ANOVA with post-hoc Dunnet's test was used. The scale bars for (A), (B) (C) show 100 μm; for (F) and (H) show 500 μm. *p<0.05, ***p<0.001 and “ns” is “non-significant”.

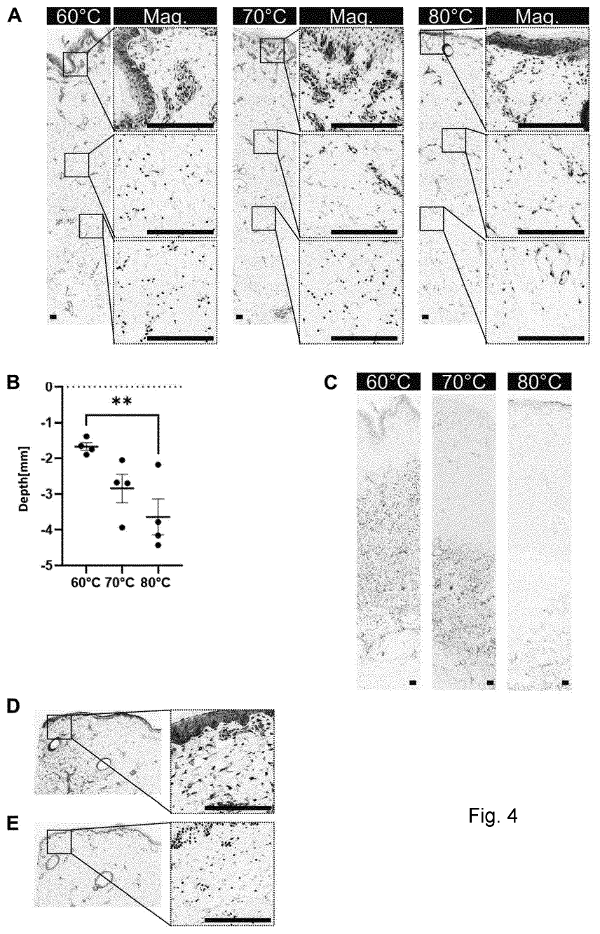

FIG. 4 shows that in burn injury the p-rpS6-zone stratifies cell death and survival responses
(A) Representative images of porcine skin biopsies collected 1.5 h after induction of 60, 70 and 80° C. burn injury. Micrographs show magnified regions of epidermis (top panel) and two dermal regions (middle and bottom panels).
(B) Quantification of the depth of termination of the HMGB1 zone in response to the 60, 70 and 80° C. burn injury at 1.5 h after the burn.
(C) Images of porcine skin biopsies representing p-rpS6 staining in 60, 70 and 80° C. burn injury.
(D) Representative image of p-rpS6 staining at the site of the excision wound.
(E) Representative image of HMGB1 staining at the site of the excision wound.

Data are from n=4 pigs per group for (B). Mean±SEM plotted.

For (B) one-way ANOVA with post-hoc Dunnet's test was used. **p<0.01. All scale bars show 100 μm.

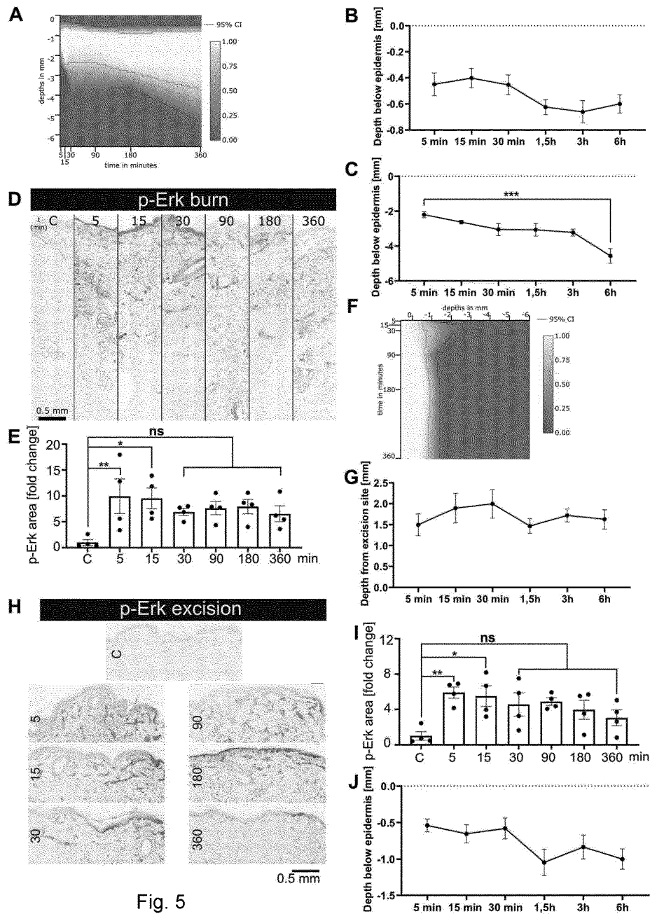

FIG. 5 shows that the induction of the p-rpS6-zone is an early-onset and long-term response
(A) Probability chart showing depth of p-rpS6 layer is samples collected 5 min, 15 min, 30 min, 1.5 h, 3 h and 6 h after induction of 60° C. burn injury in pig skin.
(B) Quantification of the initiation depth of the p-rpS6-zone in porcine skin samples collected 5 min, 15 min, 30 min, 1.5 h, 3 h and 6 h after induction of 60° C. burn injury in pig skin.
(C) Quantification of the termination depth of the p-rpS6-zone in porcine skin samples collected 5 min, 15 min, 30 min, 1.5 h, 3 h and 6 h after induction of 60° C. burn injury in pig skin.
(D) Representative images of immunohistochemical staining against p-Erk in control conditions and 5 min, 15 min, 30 min, 1.5 h, 3 h and 6 h after induction of 60° C. burn injury.
(E) Quantification of the area of p-Erk in colour-deconvolved images of control sample and at set timepoints after induction of 60° C. burn injury.
(F) Probability chart showing depth of p-rpS6 layer is samples collected 5 min, 15 min, 30 min, 1.5 h, 3 h and 6 h after induction of excision injury in pig skin.
(G) Quantification of the termination depth of the p-rpS6-zone in porcine skin samples collected 5 min, 15 min, 30 min, 1.5 h, 3 h and 6 h after induction of excision wound in pig skin.
(H) Representative images of immunohistochemical staining against p-Erk in control conditions and 5 min, 15 min, 30 min, 1.5 h and 6 h after induction of excision wound.
(I) Quantification of the colour-deconvolved images stained for p-Erk in control sample and at set timepoints after induction of excision wound.
(J) Quantification of the termination depth of the HMGB1 zone in porcine skin samples collected 5 min, 15 min, 30 min, 1.5 h, 3 h and 6 h after induction of 60° C. burn injury in pig skin.

Data are from n=4 pigs per group for all the graphs. Mean±SEM plotted. For all the graphs one-way ANOVA with post-hoc Dunnet's test was used. *p<0.05, **p<0.01, ***p<0.001 and “ns” is “nonsignificant”. All scale bars show 500 μm.

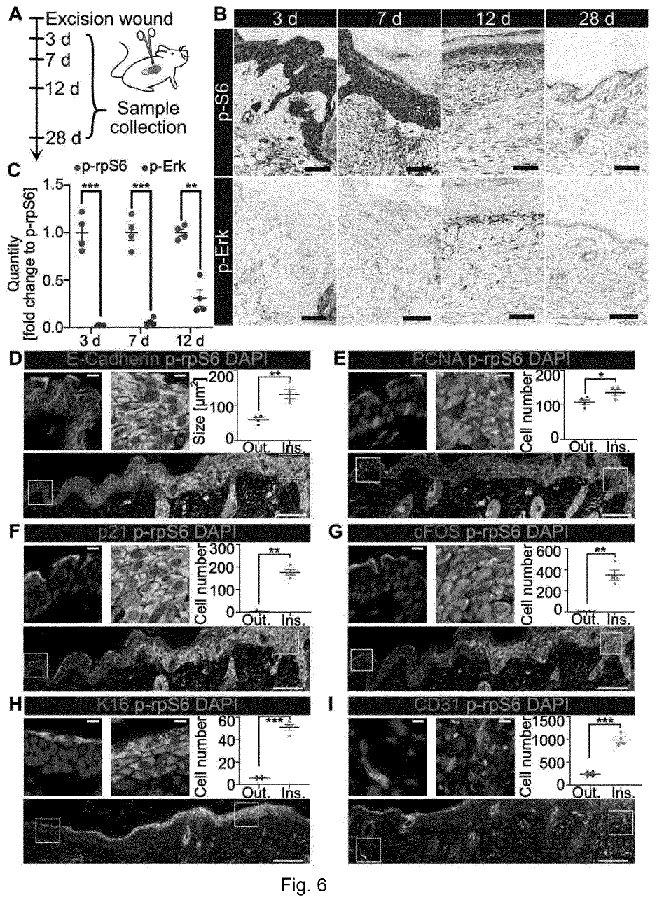

FIG. 6 shows that the p-rpS6-zone is present throughout the healing process and delineates the reach of the pro-healing cellular processes
(A) Experimental model. Mice underwent surgical removal of skin (1 cm diameter) and were sacrificed 3, 7, 12 or 28 days later.
(B) Representative images of immunohistochemical staining against p-rpS6 and p-Erk collected 3, 7, 12 and 28 days after excision injury of mice.
(C) Quantification of the colour-deconvolved images stained for p-rpS6 or p-Erk in samples collected 3, 7, 12 and 28 days after excision injury of mice.
(D) Representative images of immunohistofluorescent staining against E-cadherin (red), p-rpS6 (green) and co-stained with DAPI (blue) in a sample collected 3 days after excision injury of mice. Micrographs show regions inside or outside the p-rpS6-zone. Graph shows quantification of average size of keratinocyte present outside (Out.) or inside (Ins.) the p-rpS6-zone, with the p-rpS6-zone being immediately proximal to the wound and wound bed. 150-200 cells per animal were quantified.
(E) Representative images of immunohistofluorescent staining against PCNA (red), p-rpS6 (green) and co-stained with DAPI (blue) in a sample collected 3 days after excision injury of mice. Micrographs show regions inside or outside the p-rpS6-zone. Graph shows quantification of average number of keratinocytes positive for PCNA and present outside (Out.) or inside (Ins.) the p-rpS6-zone. 5 images per mouse were quantified.
(F) Representative images of immunohistofluorescent staining against p21 (red), p-rpS6 (green) and co-stained with DAPI (blue) in a sample collected 3 days after excision injury of mice. Micrographs show regions inside or outside the p-rpS6-zone. Graph shows quantification of average number of keratinocytes positive for p21 and present outside (Out.) or inside (Ins.) the p-rpS6-zone. 5 images per mouse were quantified.
(G) Representative images of immunohistofluorescent staining against cFos (red), p-rpS6 (green) and co-stained with DAPI (blue) in a sample collected 3 days after excision injury of mice. Micrographs show regions inside or outside the p-rpS6-zone. Graph shows quantification of average number of keratinocytes positive for cFOS and present outside (Out.) or inside (Ins.) the p-rpS6-zone. 5 images per mouse were quantified.
(H) Representative images of immunohistofluorescent staining against K16 (red), p-rpS6 (green) and co-stained with DAPI (blue) in a sample collected 12 days after excision injury of mice. Micrographs show regions inside or outside the p-rpS6-zone. Graph shows quantification of average number of keratinocytes positive for K16 and present outside (Out.) or inside (Ins.) the p-rpS6-zone. 6 images per mouse were quantified.
(I) Representative images of immunohistofluorescent staining against CD31 (red), p-rpS6 (green) and co-stained with DAPI (blue) in a sample collected 12 days after excision injury of mice. Micrographs show regions inside or outside the p-rpS6-zone. Graph shows quantification of average number of cells positive for CD31 and present outside (Out.) or inside (Ins.) the dermal compartment of the p-rpS6-zone, normalised to the area of the dermis. 14 images per mouse were quantified.

Data are from n=4 mice per group for all the graphs. Mean±SEM plotted. For the graph (C) multiple unpaired t-test was used (false discovery rate >1%). For (D-I) paired t test was used. *p<0.05, **p<0.01, ***p<0.001. Error bars for (B) and large images at (DI) show 100 μm, for the micrographs at (D-I) scale bars are 10 μm.

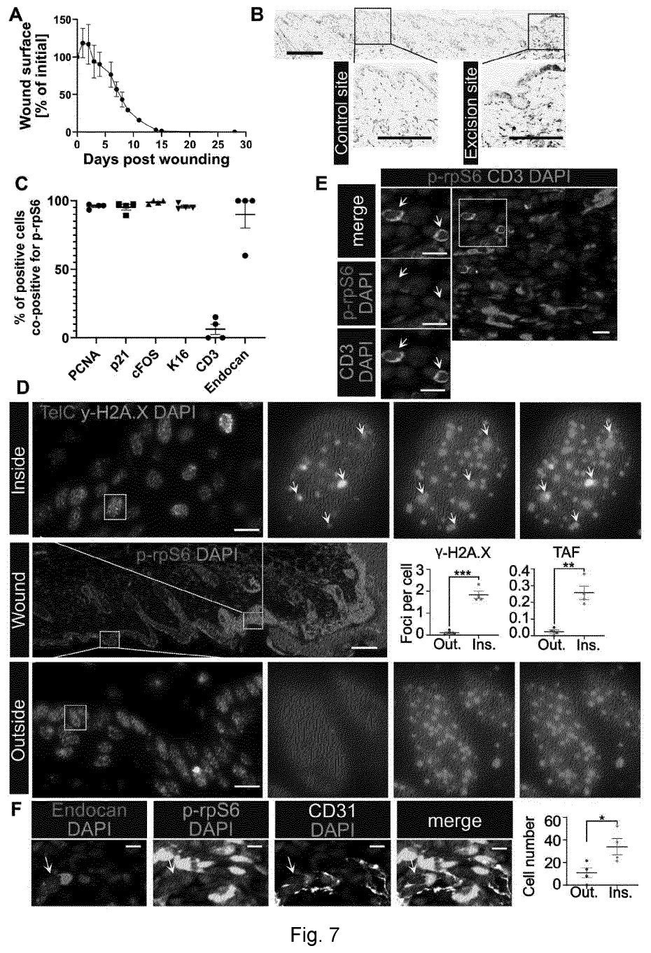

FIG. 7 shows that the p-rpS6-zone is present throughout the healing process and delineates the reach of the pro-healing cellular processes
(A) Murine excision wounds were photographed at days 0, 1, 3, 4, 6, 7 8, 9, 11, 14, 15 and 28. Wound size was normalized to the measurement from day 0 and data is shown as percentage of initial size.
(B) Representative images of immunohistochemical staining against p-Erk collected 30 min after excision injury of mouse skin.
(C) Quantification of frequency of overlap of signals from the antibody against p-rpS6 and antibodies for PCNA, p21, cFos, K16, CD3 and Endocan. Data was analyzed in 5 images per mouse for PCNA, p21, cFos, and for K16, 4-10 images per mouse for CD3 where for all imaged were taken for regions inside the p-rpS6-zone and 14 images for Endocan.
(D) Representative images of immunohistofluorescent stainings in successive sections, of the whole wound area (middle one; “wound”) against p-rpS6 (red) and co-stained with DAPI (blue) and the second the p-rpS6-zone stained against TelC (red), γ-H2A.X (green) and co-stained with DAPI (blue) showing regions inside and outside in a sample collected 3 days after excision injury of mice. Micrographs show individual cells. White arrows mark TAF. Graphs shows quantification of average number of γ-H2A.X foci and TAF per cell present outside (Out.) or inside (Ins.) the p-rpS6-zone. 6 images comprising 200 cells per mouse were quantified.
(E) Representative images of immunohistofluorescent staining against p-rpS6 (red), CD3 (green) and co-stained with DAPI (blue) in a sample collected 3 days after excision injury of mice. Micrographs show regions inside the p-rpS6-zone. White arrows mark CD3-positive cells.
(F) Representative images of immunohistofluorescent staining against p-rpS6 (green), Endocan (green) and co-stained with DAPI (blue) in a sample collected 12 days after excision injury of mice. White arrows mark an Endocan-positive cell. Graph shows quantification of average number of cells positive for Endocan and present outside (Out.) or inside (Ins.) the p-rpS6-zone. 14 images per mouse were quantified.

Data are from n=4 mice per group for all the graphs. Mean±SEM plotted for all the graphs. Scale bars for (B), the macrographs of (D) and (E) show 100 μm, for the micrographs in (D), (E) and (F) scale bars are 10 μm. *p<0.05, **p<0.01, ***p<0.001.

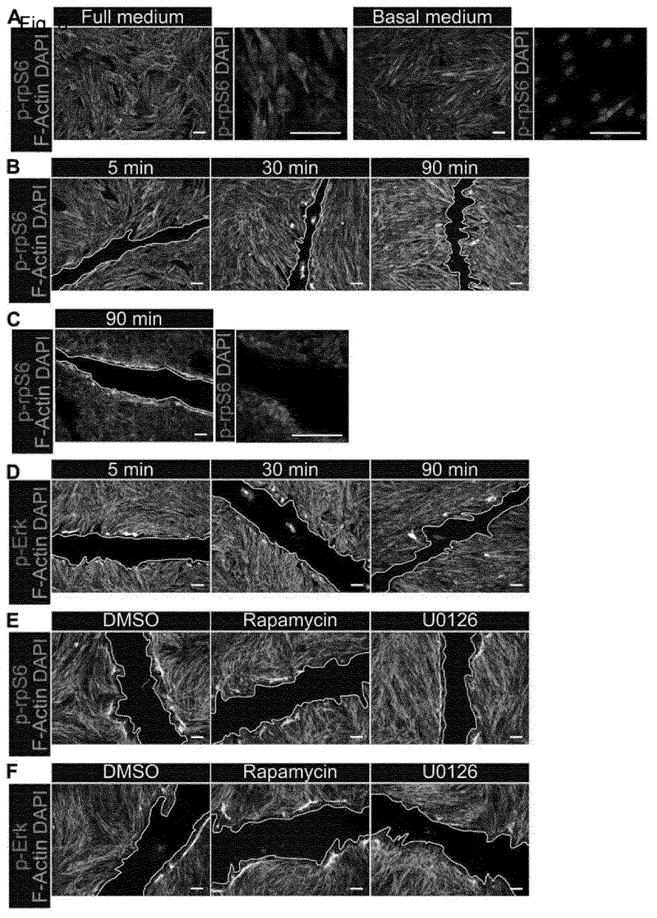

FIG. 8 shows that the formation of p-rpS6-zone is induced by DAMPs and dependent on mTOR
(A) Representative images of immunofluorescent staining against p-rpS6 (red) and co-stained with phalloidin-488 (marking F-Actin; green) and DAPI (blue) in human dermal fibroblasts (HDFs) kept in full media (containing foetal bovine serum; FBS) or basal media (without FBS).
(B) Representative images of immunofluorescent staining against p-rpS6 (red) and co-stained with phalloidin-488 (green) and DAPI (blue) in HDFs fixed 5, 30 or 90 min after induction of the scratch assay.
(C) Representative images of immunofluorescent staining against p-rpS6 (red) and co-stained with phalloidin-488 (green) and DAPI (blue) in HaCat cells fixed 5, 30 or 90 min after induction of the scratch assay.
(D) Representative images of immunofluorescent staining against p-Erk (red) and co-stained with phalloidin-488 (green) and DAPI (blue) in HDFs fixed 5, 30 or 90 min after induction of the scratch assay.
(E) Representative images of immunofluorescent staining against p-rpS6 (red) and co-stained with phalloidin-488 (green) and DAPI (blue) in HDFs treated with DMSO, Rapamycin or U0126 and fixed 30 min after induction of the scratch assay.
(F) Representative images of immunofluorescent staining against p-Erk (red) and co-stained with phalloidin-488 (green) and DAPI (blue) in HDFs treated with DMSO, Rapamycin or U0126 and fixed 30 min after induction of the scratch assay.

All scale bars show 100 μm.

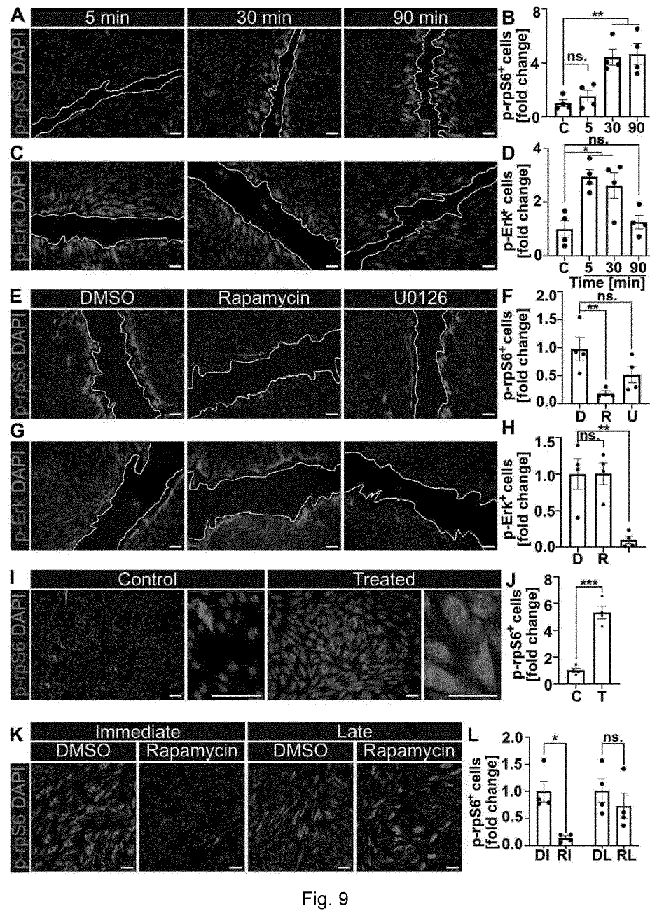

FIG. 9 shows that the formation of p-rpS6-zone is induced by DAMPs and dependent on mTOR
(A) Representative images of immunofluorescent staining against p-rpS6 (red) and co-stained with DAPI (blue) in human dermal fibroblasts (HDFs) fixed 5, 30 or 90 min after induction of the scratch assay.
(B) Quantification of the number of cells positive for p-rpS6 at 5, 30 or 90 min after induction of the scratch assay. “C” is control, that is non-scratched area. Presented as fold change over control.
(C) Representative images of immunofluorescent staining against p-Erk (red) and co-stained with DAPI (blue) in HDFs fixed 5, 30 or 90 min after induction of the scratch assay.
(D) Quantification of the number of cells positive for p-Erk at 5, 30 or 90 min after induction of the scratch assay. “C” is control, that is non-scratched area. Presented as fold change over control.
(E) Representative images of immunofluorescent staining against p-rpS6 (red) and co-stained with DAPI (blue) in HDFs treated with DMSO, Rapamycin or U0126 and fixed 30 min after induction of the scratch assay.
(F) Quantification of the number of cells positive for p-rpS6 treated with DMSO (“D”), Rapamycin (“R”) or “U0126” (“U”). Presented as fold change over control (DMSO).
(G) Representative images of immunofluorescent staining against p-Erk (red) and co-stained with DAPI (blue) in HDFs treated with DMSO, Rapamycin or U0126 and fixed 30 min after induction of the scratch assay.
(H) Quantification of the number of cells positive for p-Erk treated with DMSO (“D”), Rapamycin (“R”) or “U0126” (“U”). Presented as fold change over control (DMSO).
(I) Representative images of immunofluorescent staining against p-rpS6 (red) and co-stained with DAPI (blue) in HDFs untreated (control) or treated with media collected from mechanically lacerated cells (treated) and fixed 30 min later.
(J) Quantification of the number of cells positive for p-rpS6 at 30 min after treatment (“T”). Presented as fold change over control (“C”).
(K) Representative images of immunofluorescent staining against p-rpS6 (red) and co-stained with DAPI (blue) in HDFs treated with media collected from mechanically lacerated cells (treated) and co-treated with vehicle (DMSO) or Rapamycin before administration of media (immediate; “I”) or 30 min later (delayed; “D”). Cells were fixed 30 min after administration of the drug.
(L) Quantification of the number of cells positive for p-rpS6 at 30 min after rapamycin treatment (“R”). Presented as fold change over control (“DI”).

Data are from n=4 biological replicates (independent experiments) per group for all the graphs. For all quantifications 10 images per biological replica were used. For all the graphs mean±SEM plotted. For the graphs (B), (D), (F), (H) and (J) one-way ANOVA with post-hoc Dunnett's test was used. For (L) two-way ANOVA with post-hoc Sidak's test was used. *p<0.05, **p<0.01, ***p<0.001. All scale bars show 100 μm.

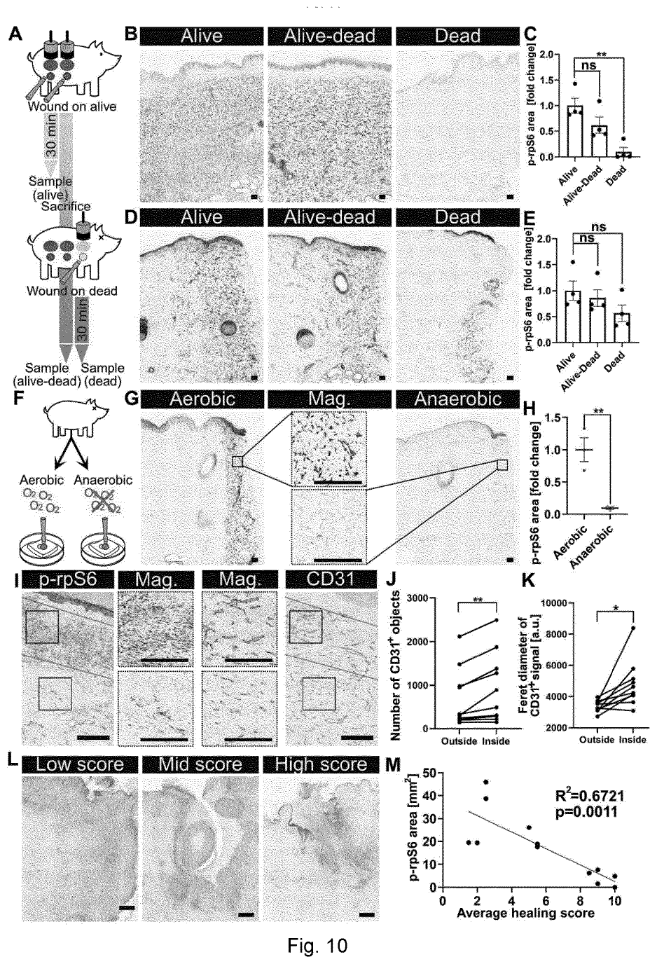

FIG. 10 shows that the p-rpS6-zone reports on the oxygen availability, vascularization and progression of healing in preclinical studies
(A) Experimental model. Pig was injured with two sets of wounds (60° C. burn, excision wound), one set of samples was collected 30 min later (condition: “Alive”). Next, the animal was sacrificed, and another injury set was performed. Samples were collected 30 min after death, from injuries performed 60 min prior on the live pig (condition: “Alive-Dead”) and from injuries performed after sacrifice (condition: “Dead”).
(B) Images of porcine skin biopsies representing p-rpS6 staining in burn samples, where the burn was performed on and collected from an alive pig (left panel), performed on a live pig and collected 60 min later from the sacrificed pig (middle panel), or performed on and collected 30 min later from the sacrificed animal (right panel).
(C) Quantification of the area of p-rpS6 in colour-deconvolved images of samples from (B).
(D) Images of porcine skin biopsies representing p-rpS6 staining around an excision wound, where the wound was performed on and collected from an alive pig (left panel), performed on a live pig and collected 60 min later from the sacrificed pig (middle panel), or performed on and collected 30 min later from the sacrificed animal (right panel).
(E) Quantification of the area of p-rpS6 in colour-deconvolved images of samples from (D).
(F) Experimental model. Porcine skin was collected shortly after sacrifice and excision wound was performed ex vivo in control (aerobic) conditions or under anoxic hood (anaerobic).
(G) Images of biopsies collected from the sides of excision wounds performed ex vivo under aerobic (left panel) or anaerobic (right panel) conditions.
(H) Quantification of the area of p-rpS6 in colour-deconvolved images of excision wounds ex vivo performed under aerobic or anaerobic conditions.
(I) Representative images of p-rpS6 (left side) and CD31 (right side) immunohistochemical stainings. The magnifications at the left and right sides of the images show the regions inside (top panels) and outside (bottom panels) of the p-rpS6-zone.
(J) Quantification of the Feret diameter of the CD31-positive objects inside the zone and in the region proximal to the zone of the same area.
(K) Quantification of the number of the CD31-positive objects inside the zone and in the region proximal to the zone of the same area.
(L) Representative images of the p-rpS6-stained immunohistochemical sections from the selected biopsies of the incision wounds collected 7 days after wounding with low (left panel), medium (middle panel) and high (right panel) score. For the high and mid scores wounds are centrally located at the section while for the low score the wound is on the right side (only partially visible at the image).
(M) Graph showing correlation between the healing score and the area of p-rpS6-positive signal.

Data are from n=4 pigs for (C) and (E) from n=3 pigs for (H). For (I)-(M) two samples were collected from each incision wound from 6 pigs at 7 days post injury resulting in n=12 biological replicates for the graph (M). Out of those, 2 samples, which were fully healed and therefore lacked a clearly defined p-rpS6-zone were excluded resulting in n=10 biological replicates for the graphs (J) and (K) per group. For all quantifications 10 images per biological replica were used. Mean±SEM plotted for (C), (E) and (H); for (J) and (K) mean per biological replica is plotted. For the graphs (C), (E) and (H) one-way ANOVA with post-hoc Dunnett's test was used; for (J) and (K) paired Student's t-test was used; for (M) Person's correlation test was used. *p<0.05, **p<0.01 and “ns” is “non-significant”. The scale bars for the images are 100 μm for (B), (D) and (G); for (I) and (L) the scale bars for the macro-images show 250 μm, while the scale bars of the magnified images represent 100 μm.

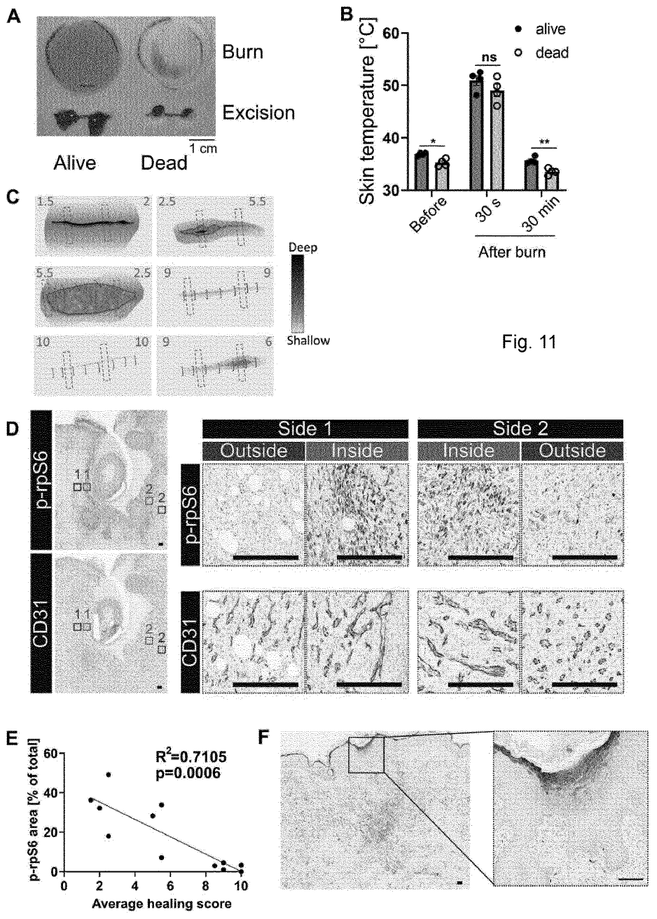

FIG. 11 shows that the p-rpS6-zone reports on the oxygen availability, vascularization and progression of healing in preclinical studies
(A) Photography of pig skin showing erythema caused by a burn injury performed when the animal was alive compared to the lack of erythema when burned at 3 min post-death.
(B) Measurements of skin temperature taken prior to burn injury, 30 s after the injury or 30 min later.
(C) Schematics that are graphical representations of the porcine incision injuries 7 days post wounding. The blue lines represent the region from which the biopsy samples were collected (and thus also the histological orientation of the subsequent sections). The number next to the line is an average of the healing score as assigned by trained veterinarians.
(D) A representative image of a single section showing heterogeneity of thickness of p-rpS6 that, however, corresponds to the degree of dermal vascularization.
(E) Graph showing correlation between the healing score and the area of p-rpS6-positive signal when normalized to the total area of the section.
(F) A representative image of an incision wound with one of the highest healing scores that still shows remnants of p-rpS6 in epidermis and dermis even after the apparent macroscopical completion of the healing process.

Data are from n=4 pigs for (B). Two samples were collected from each incision wound from 6 pigs at 7 days post injury resulting in n=12 biological replicates for the graph (E). Mean±SEM plotted for (B). For the graph (B) two-way ANOVA with post-hoc Sidak's test was used. For the graph (E) Person's correlation test was used. The scale bar for the skin photographs (B) is 1 cm and for images (D) and (F) it is 100 μm. *p<0.05, **p<0.01 and “ns” is “non-significant”.

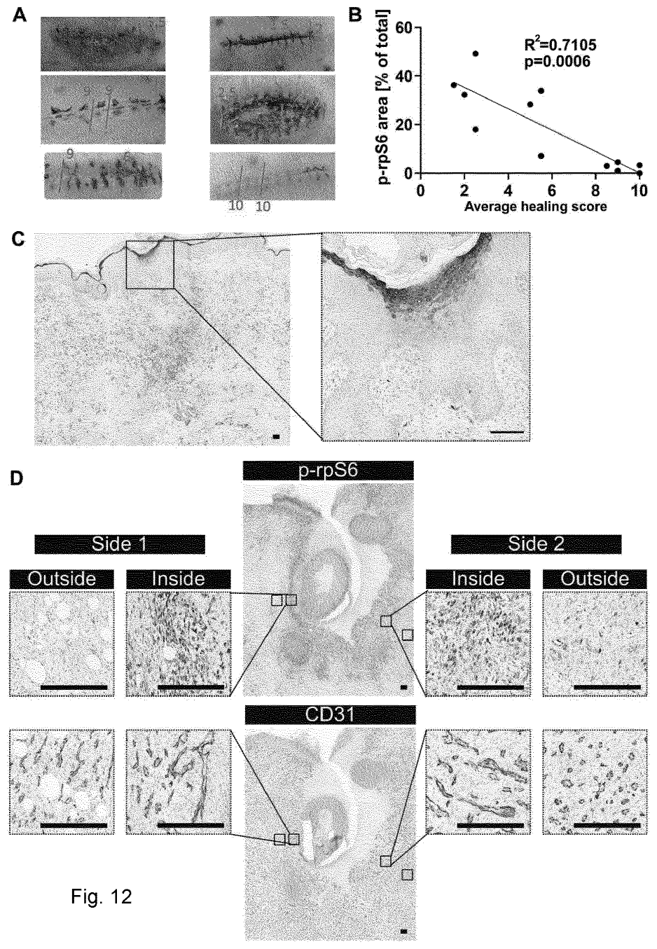

FIG. 12 shows that the p-rpS6 zone reports on the progression of healing and delineates highly vascularized regions in preclinical studies
(A) Schematics that are graphical representations of the porcine incision injuries 7 days post wounding. The blue lines represent the region from which the biopsy samples were collected (and thus also the histological orientation of the subsequent sections). The number next to the line is an average of the healing score as assigned by trained veterinarians.
(B) Graph showing correlation between the healing score and the area of p-rpS6-positive signal when normalized to the total area of the section.
(C) A representative image of an incision wound with one of the highest healing scores that still shows remnants of p-rpS6 in epidermis and dermis even after the apparent macroscopical completion of the healing process.
(D) A representative image of a single section showing heterogeneity of thickness of p-rpS6 that, however, corresponds to the degree of dermal vascularization.

All scale bars represent 100 μm.

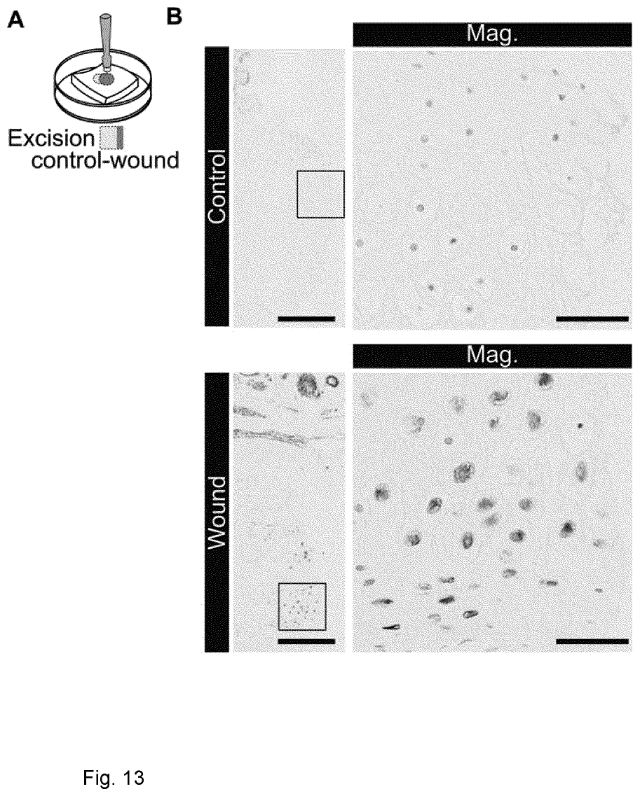

FIG. 13 shows that p-rpS6 can be used to visualize damage/wounding of cartilage: p-rpS6 is induced upon damage of cartilage. (A) Sheep ear ex vivo was damaged by an excision wounding using a biopsy puncher. After 1.5 h samples were collected from the damaged and control (unwounded) sites. (B) The representative histological images show p-rpS6 staining of unwounded ear cartilage (top image) and the wounded site of the cartilage (bottom image). The scalebar is 100 μm.

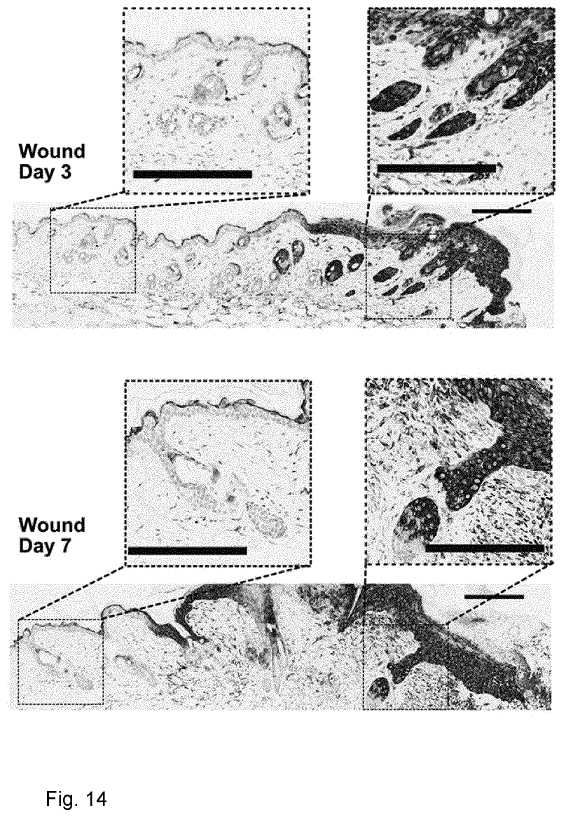

FIG. 14 shows that the hair follicles surrounding the wound (but not those that are distant from wounds) and found these to be positive for p-rpS6 (all scale bars are 100 μm).

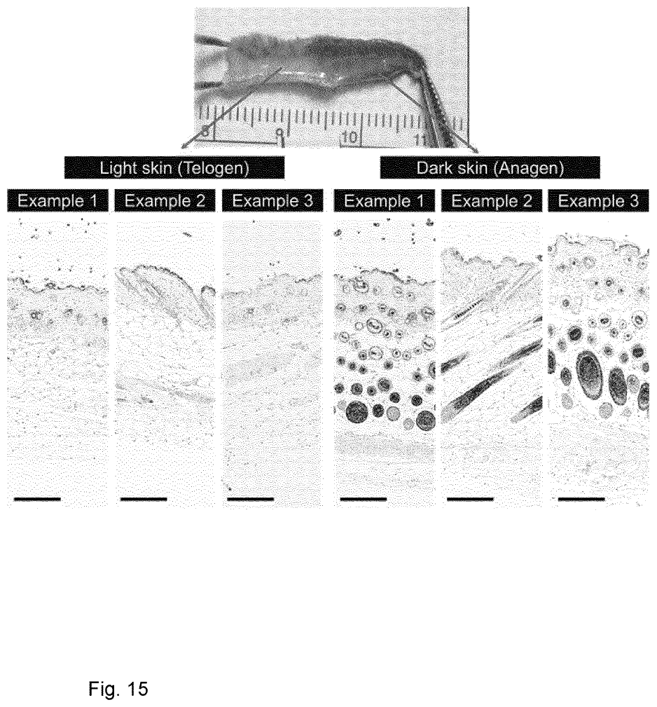

FIG. 15 shows that p-rpS6 can be used to visualize hair growth and functionality of hair follicles.

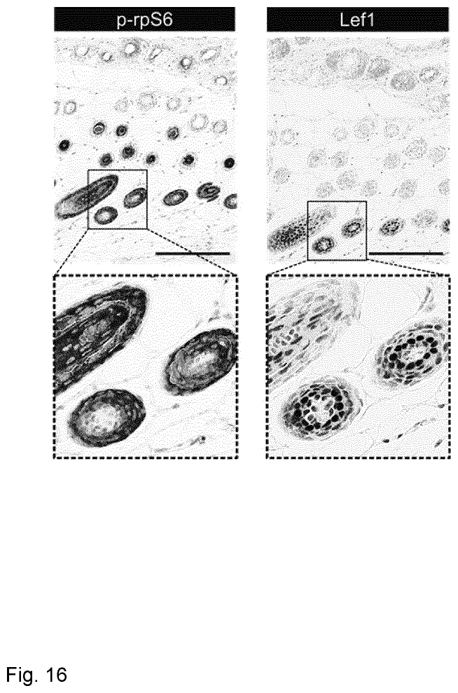

FIG. 16 shows that anagen hair follicles of homeostatic skin are positive for both Lef1 and p-rpS6.

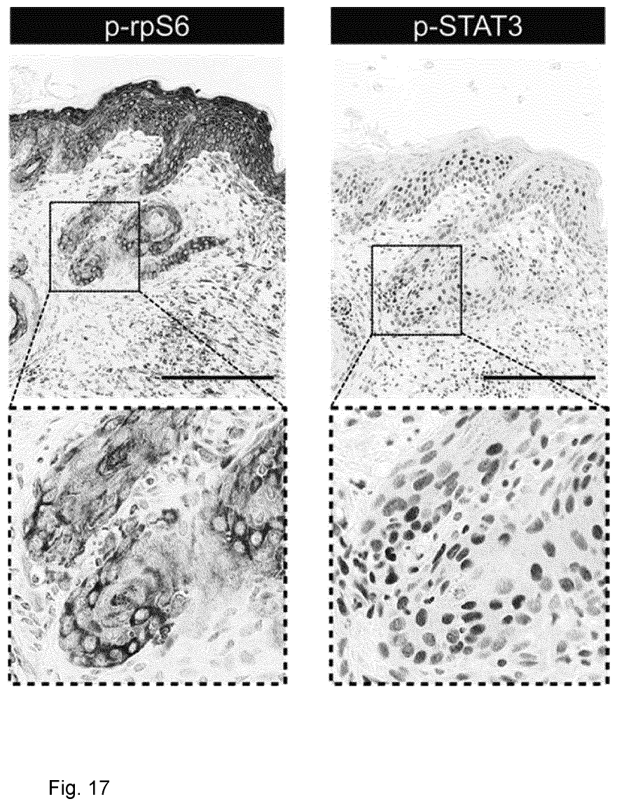

FIG. 17 shows that STAT3 signaling has been related to growth and function of hair follicles and that the follicles proximal to the wound site are positive for both p-rpS6 and p-STAT3 Y705.

### EXAMPLES

### Methods

### Ex Vivo and In Vivo Wounding Experiments in Pigs

Pig experiments were performed according to the ethical guidelines by the host institution with support of the veterinarian team of the LBI Trauma. All experimental protocols were approved in advance by the Municipal Government of Vienna in accordance with Austrian law and the Guide for the Care and Use of Laboratory Animals as defined by the National Institute of Health (revised 2011). These protocols allow for non-recovery experiments with biopsy and sample collection up to 6 h post burn. Samples were collected from a total of 26 male pigs.

Pigs were anesthetized using zoletil (250 mg of tiletamine with 250 mg of zolazepam) mixed with 100 mg of xylazine in 5 ml of solution—of this 1-2 ml were administered intramuscularly per 15 kg of body weight. A catheter was then placed in the lateral ear vein and 2 mg/kg propofol (10%) were administered intravenously. Pigs were then intubated and ventilated. During the procedure pigs were kept under inhalation anesthesia (oxygen, sevoflurane (3-6%)). During the procedure pigs were kept under inhalation anesthesia (oxygen, sevoflurane (3-6%)). A pulse oximeter (SpO₂, heart rate), and temperature probe (esophageal) were additionally used for monitoring animals. In addition, the pigs received intravenous 2-10 ml/kg bw Elomel Isoton® (Fresenius Kabi, Graz, Austria), 0.008 mg/kg/h sufentanil, and 2.5 mg/kg/h rocuronium bromide while under anesthesia. During the surgical procedure, the sufentanil dose was adjusted according to the condition of the animals and close attention was paid to provide adequate analgesia. If blood pressure dropped, 5 pig/kg/h L-norepinephrine hydrochloride was administered intravenously until effect (MAP 60>mHg). Operation logging was performed every 15 min. Further instrumentation of the animals consisted of preparation of the carotid artery (monitoring of central arterial blood pressure) and jugular vein (infusion, administration of analgesic and muscle relaxant) and placement of a bladder catheter. Subsequently, wounds were applied to the shaved dorsal skin. Wounds were induced by burn (standardized thermal lesions of grade I or grade IIa), biopsy punch (as a proxy for excision wounds) or needle prick (29 G U-40 insulin needle). For burns, an aluminum block with a diameter of 3 cm on a specially designed plunger was heated to 60, 70 or 80° C. in a water bath, the temperature of the surface of the block was measured using an infrared temperature gun, and the plunger was pressed with a constant and normalized across experiments pressure of approximately 0.4 kg/cm² onto the side of the pig as established (Branski et al., Burns 34 (2008), 1119-1127). Samples were collected at specified time points after the burn, specifically 5, 15 and 30 min and 1.5, 3 and 6 h. Excision wounds were modelled by performing a 6 mm biopsy punch of the skin, removing the biopsy and leaving a 6 mm diameter wound. The edges of this wound were then collected at the same time points as for the burn model. Samples were immediately fixed in 10% formalin for 24 h at room temperature in histology cassettes. They were then washed carefully for 1 h with water, incubated for 1 h in 50% ethanol and stored in 70% ethanol until the samples were finally embedded in paraffin.

For the experiments focused on hypoxia, two sets of burn and excision wounds were performed on the dorsal skin of an alive and anesthetized pig. The first set of samples was collected after 30 min, after which the pig was sacrificed. The second set of samples was collected 30 min after death. Before that, approximately 3 min after death, a third set of burn and second excision wounds was placed near the first two, and samples were collected after 30 min. For this set of experiments, temperature measurements of the skin were taken using an infrared temperature gun immediately prior to the burn, the temperature of the burned skin was measured immediately after the burn and again at sample collection to control for fluctuations in body temperature caused by death.

For ex vivo experiments, dorsal porcine skin was removed from the sacrificed animal and kept for acclimatization for 1 h in an incubator at 37° C. The tissue was then subjected to wounding using a 6 mm biopsy punch, as described above.

To analyze the effect of an anoxic environment, ex vivo skin was transported in a warmed box to the anoxia chamber. One piece of skin was wounded and left to recover in an incubator at 37° C. in atmospheric oxygen, while the second piece was placed inside the anoxic laminar hood for wounding and then left to recover in the anoxic incubator at 37° C.

### In Vivo Experiments in Wild-Type Mice

Mouse experiments were performed according to ethical guidelines of the host institution with support of the veterinarian team of the LBI Trauma. All experimental protocols were approved in advance by the Municipal Government of Vienna in accordance with Austrian law and the Guide for the Care and Use of Laboratory Animals as defined by the National Institute of Health (revised 2011). Female Balb/C mice were purchased from Janvier labs. 20 mice were used for experiments at an age of 12 weeks and an average weight of 20-25 g. Five animals were housed per Type-III cage on a 12 h light-dark diurnal cycle with room temperature between 21 and 23° C. Standard rodent diet (Abbedd Lab & Vet Service, Vienna, Austria) and water were provided prior to and throughout the experiments. All surgical procedures and treatments were done under inhalation anesthesia using an oxygen/sevoflurane mixture (4-5%) (Sevorane®, AbbVie Inc., North Chicago, Illinois, USA). Analgesia (1 mg/kg of meloxicam p.o.) was administered 120 min prior to surgery and post-operatively daily until end of study or wound closure.

On the day of the wounding procedure, mice were anesthetized, the hair was shaved in the dorsal area and the skin was disinfected with Isozid®. Subsequently, one full thickness puncture wound was administered in the posterior dorsal region. A template was used to draw a 1 cm diameter circle and the skin was then carefully cut along this circle using scissors to avoid damaging the underlying muscle fascia. After wounding, mice were placed in a cage with a 37° C. heating pad until recovery. Then the mice were transferred back to their cages and housed for the duration of the study. Animals were monitored daily for any signs of infection, pain and loss of weight. To follow the healing process, digital photos were taken using a camera (LifeViz micro, Quantificare, France). Wound area was analyzed by a planimetric measurement with a free-hand tool using Fiji image processing software (ImageJ, National Institute of Health, USA; Schindelin et al., Nat. Methods 9 (2012), 676-682). Mice were sacrificed at the end of the observation period under deep sevoflurane anaesthesia via cervical dislocation at 30 min, 1.5 h, 3 d, 7 d, 12 d and 28 d after wounding. Each wound was collected with the surrounding tissue and placed in histology cassette. The tissues were fixed in 10% formalin for paraffin sections.

### Ex Vivo Experiments in Human Skin

The skin samples for the sections used in this study were approved by the Ethics Committee of the Medical University of Vienna (1969/2021), and written informed consent was obtained from all subjects. Tissue was used for experiments within 5 hours from removal, throughout which the tissue was stored at room temperature. Prior to the experiment the tissue was acclimatized in an incubator at 37° C. and wounded using the same procedure applied to ex vivo pig skin.

### Histology

For sample analysis both immunohistochemistry and immunohistofluorescence were used. Skin samples were fixed in 10% buffered formalin for 24 h and, embedded in paraffin and sectioned at 4 μm. Sections were deparaffinized and rehydrated through a graded alcohol series. For heat-induced antigen retrieval samples were incubated in a 0.1 M Tris with 0.01 M EDTA (pH 9.0) or sodium citrate buffer 0.01 M (pH 6.0) in a steamer at 95° C. for 20 min, or in the microwave where the buffer was brought to a boil and then left for 15 min at sub-boiling temperature.

For immunohistofluorescence, samples were blocked in PBS containing 0.4% BSA (Sigma Aldrich) and 1.6% normal goat serum (Vectorlabs) for 1 h at room temperature. Samples were then incubated with appropriate primary antibodies (Table 1) in blocking buffer overnight at 4° C. Slides were washed three times with TBS-Tween (TBS-T) and incubated for 1 h with DAPI and fluorescent secondary antibody (Thermo Fisher Scientific, Waltham, MA, USA) and mounted in MOWIOL mounting media.

For immunohistochemistry, samples were blocked for 10 min using BLOXALL (Vectorlabs), were washed in TBS-T and were then incubated for 1 h at room temperature of overnight at 4° C. with primary antibody (Table 1). After incubation, samples were washed three times, incubated with HRP-conjugated secondary antibody (BrightVision) for 30 min and stained for 6 min with NovaRed (Vectorlabs). Finally, samples were counterstained, dehydrated and mounted. All applied primary antibodies can be seen in Table 1 and were used in dilutions recommended by the manufacturer.

To analyze immunohistochemistry stainings, the amount of the bound antibody was quantified using ImageJ. Specifically, slide-scans were exported to the tif format, and the number of positive pixels was quantified in experiment-specific regions of interest (ROI) using “Colour deconvolution” and vectors for “H DAB” followed by signal thresholding and “Analyze Particles” function to determine the stained area. All the settings were consistent for the samples within a given experiment.

The analysis of frequency and size of CD31-positive objects in relation to p-rpS6 for porcine and human samples was done by first aligning histological images in Fiji28 using the moving least squares plugin. Between 10 and 20 points which could be identified on both images were marked using the multi point selection tool. The CD31 stained image was warped to match the ps6 staining. Due to the size of the images, the RGB colour channels were split into three separate images, warped individually using the moving least squares plugin with the “rigid” method and then merged again into a single RGB image. Alignment errors were corrected by moving or adding points and repeating the warping procedure. The aligned images were then processed as described above. P-rpS6-positive area was selected and the ROI was copied onto the CD31-stained section. In this way two images of CD31 staining were obtained, one of the inside of the p-rpS56 zone and a second for which the same ROI was moved outside of the p-rpS6-zone (thus the selected areas are identical in shape and size). Both images were colour-deconvolved, thresholded for positive signal and analyzed using “Particle analysis” tool in ImageJ deriving the number (“Count”) and size (“Feret”) of the CD31-positive objects inside and outside of the zone.

The histological analysis based on the assessment of depth of the initiation of the signal was performed in OlyVIA software (Olympus Corporation, Tokyo, Japan). For this analysis several measurements of the length between epidermis and the positive area were made and the average compared between animals and conditions.

### Depth Probability Chart

For each timepoint after burn or excision wound, mean and standard deviation were calculated using Google Sheets (Google LLC). The depths were assumed to follow a normal distribution and were linearly interpolated between measured timepoints using the “Forecast. Linear” function. Probabilities for each timepoint and depth were calculated using the cumulative “Normdist” function. Confidence interval for each timepoint was calculated using the “Confidence” function.

To convert the calculated probabilities into an image, probabilities for each timepoint and depth were exported as a tab separated text file. Using Fiji, an empty 32-bit image was created and each pixel assigned the probability value in the tab separated text file using a macro. Colour values were assigned to each probability using a lookup table.

### Needle 3D Zone Image and its Cross-Sectional View

To create the 3D segmentation and virtual cross section, serial histological images were aligned using Fiji28. First, distortions of the individual histological slides were corrected by creating point ROIs which marked the position of the same 7 hair follicles on each slide. Using the moving least squares plugin, the slides were distorted so the position of the hair follicles was identical for all slides. This procedure removes distortions from the cutting and mounting of the slides, but since not all follicles run parallel, some misalignment remains. To correct this, images were rigidly registered by drawing a line between two follicles which lie on opposing sides of the defect and are approximately equidistant. The line was then moved so the centre of the line lies in the centre of the defect. Images were then rotated so the angle of the lines for all slides matches the line on the first slide and translated so the centre of the line is in the same position for all slides. Registered images were combined into a single stack.

The outline of the affected area was marked using the lasso tool and saved with the ROI manager. Segmentations for the skin and affected area were saved as binary images. 3D rendering was created using CTVox (Bruker Corporation, Billerica, MA, USA). To create the virtual cross section, a rectangular selection with a height that matches the slice distance was placed over the centre of the defect. The stack was then cropped to this selection and a montage was created where all slices in the cropped stack are arranged in a single column.

### In Vitro Experiments

To model a skin wound in vitro, cell cultures were subjected to a damage assay to monitor cellular response mechanisms. Commercially available human dermal fibroblasts (HDFs) were cultured in HDF medium (DMEM/F12 (Sigma Aldrich), 10% fetal bovine serum (Sigma Aldrich) and 4 mM L-Glutamine (Gibco)), subcultivated at 90% confluency and used for experiments below passage 15. Immortalised human keratinocytes (HaCaT) were maintained in HACAT medium (DMEM high glucose (Sigma Aldrich), 10% fetal bovine serum (Sigma Aldrich) and 2 mM L-Glutamine (Gibco) and subcultivated at 60% confluency. All cell culture experiments were performed in a humidified incubator at 37° C. and 5% CO₂.

The scratch assays were performed on cells seeded 7,000 cells/cm² (HDF) at 10,000 cells/cm² (HACAT) and grown for 5 to 7 days until confluency. 24 h prior to scratching, medium was exchanged to basal medium (DMEM/F12 or DMEM without any supplements) to eliminate endogenous activation of signaling pathways. For signaling pathway manipulation, cells were treated using either vehicle (0.1% DMSO), 20 nM of mTOR inhibitor rapamycin, or 10 μM of MEK inhibitor U0126 for 10 to 30 min prior to scratch or 30 min after administration of the media with DAMPs. For the scratch assay, the confluent cell layers were then subjected to damage by scraping a blunt-edged 21 g needle through the confluent cell layer, thereby creating a standardized scratch with broken cells on either edge. All scratches were verified using an inverted microscope directly after scratch. After 5, 30 or 90 min of incubation the cell layers were fixed using 10% formalin for 10 min, washed, and stored at 4° C. until they were stained.

For immunofluorescence staining, the coverslips containing damaged and control cells were permeabilized using 0.5% Triton X100 for 5 min, washed three times with PBS for 5 min each, and blocked using PBS with 5% goat serum (VECS-1000, Vector Laboratories) for 1 h. Subsequently, primary antibody and PhalloidiniFluor 488 (Abcam, ab176753) were applied in optimized dilution and incubated overnight at 4° C. After primary antibody application, the coverslips were washed three times for 5 min and secondary antibody and DAPI were applied for 1 h, cells were washed, and mounted on slides using MOWIOL solution. For quantification of rpS6 and Erk phosphorylation, ten images per slide were taken along the scratch using an inverted microscope Nikon (Nikon Corporation, Tokyo, Japan) with set exposure times. These images were processed and quantified for total cell count and activated cell count (p-rpS6 or p-ERK positive cells) using set thresholds and particle analysis in Fiji. All applied primary antibodies can be seen in Table 1 and were used in dilutions recommended by the manufacturer.

TABLE 1
Primary antibodies

| | Host | Catalogue | |
|---|---|---|---|
| Antigen | species | Number | Company |
| a-SMA | mouse | A2547 | Sigma |
| CD3 | rat | MCA1477 | Biorad |
| cFos | mouse | ab208942 | Abcam |
| Cleaved Caspase 3 | rabbit | 9661 | Cell Signaling |
| E-Cadherin | mouse | M3612 | Dako |
| γ-H2A.X | rabbit | 9718 | Cell Signaling |
| Gapdh | mouse | MA5-15738 | ThermoFisher |
| Hmgb1 | rabbit | ab18256 | Abcam |
| p-Erk (Thr202/Tyr204) | rabbit | 4370 | Cell Signaling |
| p-rpS6 (S235 + S236) | rabbit | 4858 | Cell Signaling |
| p-rpS6 (S240 + S244) | rabbit | ab81081 | Abcam |
| p21 | rabbit | ab109520 | Abcam |
| PCNA | mouse | ab29 | Abcam |
| (Total) rpS6 for western blot | rabbit | 2217 | Cell Signaling |
| (Total) rpS6 for histology | Mouse | 2317 | Cell Signaling |
| Endocan | Goat | AF1999 | Novus Biologicals |
| CD31 in porcine histology | Mouse | Sc-376764 | Santa Cruz |
| CD31 in mouse histology | Rat | DIA-310 | Dianova |
| K10 | guinea pig | GP-K10 | Progen |
| K16 | guinea pig | GP-K16 | Progen |

### Western Blotting

Skin biopsies were snap frozen in liquid nitrogen until processing. Tissue was then pulverised using a mortar and pestle while submerged under liquid nitrogen. Tissue powder was lysed using RIPA lysis buffer (Sigma-Aldrich, 20-188) supplemented with protease and phosphatase inhibitors (Sigma-Aldrich, P8340 and P0044), 0.2% SDS and 0.5 mMv DTT. Samples were lysed for 1 h on ice, then sonicated 3 times at 0.5 kJ per pulse and finally centrifuged 4 times at 12,000×g for 10 min to remove all fat from the lysate. Samples quantified using the Pierce BCA protein assay kit, boiled in NuPAGE LDS Sample Buffer (Thermo Fisher) and run on Bolt 100 Bis-Tris Plus Gels, 12-well gels (Thermo Fisher) in MOPS buffer. The protein was transferred to a PVDF membrane using the Bradford Trans-blot Turbo Transfer system for protein detection. Membranes were incubated in primary antibody overnight, then incubated with secondary HRP-conjugated antibody for 1 h and developed using ECL (Thermo Fisher). GAPDH was used as a normalisation control.

### Statistical Analysis

All statistical analyzes including testing the normality of data distribution were performed using GraphPad Prism 9.3.1 and a P value<0.05 was considered as significant. For differences between 2 groups paired or unpaired two-tailed t-test was used, data were further tested for equality of variances using F test. For >2 group comparisons, one-way ANOVA with Dunnett's multiple comparison test was used. For analysis concerning more than one variable two-way ANOVA with Sidak's multiple comparison test or multiple t test was used. Correlations were assessed using Pearson's rank correlation test.

### Results:

RpS6 Phosphorylation Marks a Zone of Tissue Activation in Response to Wounding, a Damage Response which is Conserved in Mammals

Wounding leads to cell and tissue damage, and eventual healing, but the molecular connections between wounding and healing are not well understood. The combination of damaging stimuli and factors causing cell expansion (growth, increase in metabolism and proliferation) can lead to heterogenous cellular responses such as cell death or senescence. This concept inspired us to assess changes in the quantity of p-rpS6 level, which is often associated with an increase in the metabolic activity of cells upon wounding and during healing.

We first investigated several injury models in juvenile pigs as porcine skin is highly similar to human skin. Specifically, in a burn injury model the skin of animals was wounded using a metal block pre-heated to 60° C. (similar to our previous studies; Branski et al., Burns 34 (2008), 1119-1127), excision injury was performed by taking a 6 mm punch biopsy and finally a prick injury was induced by a fine 29 gauge needle (FIG. 1 A). In unwounded control skin the phosphorylation of rpS6 at the sites S235/236 was present solely in the stratum granulosum of epidermis and in some hair follicles (FIG. 1 B). In contrast, at 1.5 h after injury there was a dramatic increase in the amount of p-rpS6 affecting all layers of the skin and cells including keratinocytes, fibroblasts and endothelial cells (FIG. 1 B-H). The signal was visible as an approximately 2 mm thick band matching the orientation of the damage: burn injury led to a horizontal activation band, excision injury led to a vertical one, while needle injury led to a cylindrical one (consequently smaller in size than for the excision or burn injury) (FIG. 1 B-H). In order to visualize the dimensionality of the zone in the burn injury, we collected a large skin sample from the edge of the burned area and the subsequent analysis revealed a zone positive for p-rpS6 surrounding the site of the thermal damage (FIG. 1 E; FIG. 2 A). In order to visualize the zone around the needle prick sequential tissue slices were cut starting at the epidermis, then stained and reassembled to display the lateral view and the 3D projection of the zone (FIG. 1 H). Finally, we confirmed the increase in p-rpS6 by using an antibody against an alternative phosphorylation site of rpS6 (serines 240/244) (FIG. 2 B). Moreover, we were able to validate the increased phosphorylation of rpS6 at serines 235/236 in protein lysate from control, burned and excision-wounded skin by western blot (FIG. 2 C). This shows that formation of the p-rpS6 zone is a common feature of at least several, different types of skin injury: burns, excision and needle injury.

To assess how reliable and evolutionarily-conserved the induction of the p-rpS6-zone is, we used murine skin injured by full-thickness excision wound. Mice were sacrificed 0.5 to 1.5 h after injury (FIG. 1 I). Matching our observations in porcine skin, we found a thin layer of p-rpS6 limited to the stratum granulosum of unwounded skin, while wounded murine skin displayed a broad activation of rpS6 (FIG. 1 J). Similar to porcine skin, the p-rpS6-zone in murine skin was approximately 1.5 mm wide and encompassed a variety of cell types in all skin layers (FIG. 1 J, K; FIG. 2 D).

Finally, to determine the value of these findings for humans, we used human skin collected as a by-product of surgical procedures (FIG. 1 L). We performed excision wounds using a 6 mm punch biopsy in the skin within an ex vivo setting and again found a strong induction of the p-rpS6-zone (FIG. 1 M, N; FIG. 2 E), which confirms evolutionary conservation of this response across mammals (FIG. 1 A-K).

In Burn Injury the p-rpS6-Zone Stratifies Cell Death and Survival Responses

Clinical practice involves the assessment of wound severity using measurements of cell death, predominantly necrosis and apoptosis. However, to our knowledge there are currently no reliable tissue markers that could be used to describe the range of tissue response to wounding without relating directly to cell death. Intrigued by our earlier observation that the p-rpS6-zone starts immediately adjacent to the damage in an excision wound but it is shifted to 2 mm below the skin surface in burn wounds (FIG. 1), we investigated about the relationship between p-rpS6 induction and cell death. Thus, we further investigated if cell death by heat exposure might be responsible for the different location of the zone in burn wounds.

We used two well established markers of cell death: high mobility group box 1 (HMGB1)—a protein which leaks out of the nucleus to the cytoplasm in response to cell stress and necrosis, and cleaved caspase 3—a marker of apoptosis. We found that at 1.5 h after a 60° C. burn, the cells in the top 2 mm of the skin displayed HMGB1 leakage while the HMGB1 content of the cells below that layer remained unaffected (FIG. 3 A, D). Cells positive for cleaved caspase 3 were present in a thin layer of approximately 0.5 mm width that overlapped with the lower part of the necrotic layer (FIG. 3 B, D).

Overall, the p-rpS6-zone starts with the beginning of the apoptotic layer, but also stretches deeper into the dermis where we observed no markers of cell death (FIG. 3 C, D). With higher temperatures of 70 and 80° C., we observed a downward shift of both the HMGB1 layer (FIG. 4 A, B) and the p-rpS6-zone (FIG. 3 E; FIG. 4 C) by ~1 mm per 10° C. suggesting that this stratification relates directly to burn-induced skin response. In contrast, 1.5 h after excision wounding, we observed no evident leakage of HMGB1 adjacent to the wound suggesting an insignificant amount of cell death, while the p-rpS6-zone was strongly present (FIG. 4 D, E). This shows that the p-rpS6-zone appears in response to damage without being specific to dying cells. These results demonstrate that the p-rpS6-zone is a novel and powerful marker of tissue response to wounding. The p-rpS6-zone constitutes the first early-onset marker informing of tissue response to injury that goes beyond cell death.

The Induction of the p-rpS6-Zone is an Early-Onset and Long-Term Response

With the majority of markers related to tissue wounding—such as infiltration of immune cells—appearing hours to days after induction of damage, we determined how rapidly and stably p-rpS6 is induced in excision and burn injuries. One exception is the Erk signaling pathway, which was recently found to be rapidly induced in response to wounds in invertebrates and fish. In order to determine the time of induction and the propagation dynamics of p-rpS6 and p-Erk we analyzed samples at minutes to hours after burn or excision injury in a porcine model (FIG. 3).

Burn injury resulted in the appearance of the p-rpS6-zone within minutes and reached statistical significance at 15 min after wounding (FIG. 3 F, G). Moreover, the intensity of the signal plateaued and remained present in high quantity and of significant intensity even at 6 h post-injury (FIG. 3 F, G). When analyzing the depth of initiation and termination of the p-rpS6-zone, we did not observe any significant changes in the depth of initiation within 6 h after injury (FIG. 3 F; FIG. 5 A, B). However, we did observe a gradual propagation of the terminus of the p-rpS6-zone, which penetrated deeper into dermis over time, an effect especially visible at 6 h (FIG. 3 F; FIG. 5 A, C).

Similar to p-rpS6, p-Erk was detectable very early after burn injury, but unlike p-rpS6, the level of p-Erk showed a sharp and significant rise as early as 5 min after injury (FIG. S3 D, E). However, the rapid increase was followed by an equally rapid decrease and 30 min after burning the level of p-Erk was no longer significantly different from control (FIG. 5 E) showing an acute induction of p-Erk with a rapid decline. Unlike p-rpS6, which showed a clear death- and damage-related stratification in signal (FIG. 1, 3), the p-Erk signal did not present a distinct layer of response and no obvious differences in signal spreading or propagation over time (FIG. 5 D).

Consistently, excision wounds also showed an early and long-lasting induction of p-rpS6 reaching significance at 5 min after wounding and remaining elevated throughout the entire monitoring period (FIG. 3 H, I). In contrast to burn injury, however, we did not detect any significant changes in the spatial positioning of the p-rpS6-zone, which stably marked a layer of around 2 mm from the side of the excision wound (FIG. 3 H; FIG. 5 F, G).

Just as in burns, the significant increase of p-Erk signal in excision wounds was detectable as early as 5 min after injury (FIGS. 5 H, I). However, in contrast to p-rpS6 the statistical significance was lost for timepoints collected after 30 min of injury and unlike p-rpS6, we did not observe any consistent spatial relationship to the wound site (FIG. 5 H, I).

While HMGB1 leakage occurred as early as 5 min after induction of the burn wound, there was no significant propagation of the necrotic zone within the time period of up to 6 h post injury (FIG. 5 J). This shows that the p-rpS6-zone appears quickly and concomitantly with the earliest markers of cell death; taken together with its much wider area of induction it represents a fast and easy to perform technique to detect and quantify the damage response on a tissue scale.

Our results univocally demonstrate that the p-rpS6-zone is an early-response and stable marker of tissue injury. As our results on the rapid appearance of the p-rpS6-zone come from in vivo porcine skin wounds as well as ex vivo human skin wounds, these findings are directly translatable to humans.

The p-rpS6-Zone is Present Throughout the Healing Process and Encompasses Cellular Processes Associated with Healing

The process of wound healing is associated with a variety of histological features including an increased proliferation and size of keratinocytes, induction of cellular senescence, expression of proto-oncogenes such as c-Fos, infiltration of immune cells and angiogenesis. However, the common molecular traits connecting these features are not well established.

In order to investigate the relevance of the p-rpS6-zone in the healing process we analyzed murine skin samples at 3, 7, 12 and 28 days after wounding following the scheme shown in FIG. 6 A. In our experiment, wounds were re-epithelialized by day 12 and completely healed by day 28 (FIG. 7 A). The p-rpS6-zone was detectable throughout the process of healing and the p-rpS6 signal was present in keratinocytes forming the epithelial tongue, in dermal fibroblasts proximal to the injury site and re-growing blood vessels (FIG. 6 B). The signal diminished after completion of the healing process with a loss of staining in both the epidermis (present solely in the stratum granulosum and hair follicles) and the dermis and associated structures (FIG. 6 B), returning to control levels (FIG. 1). We observed that just as in the case of p-rpS6 (FIG. 1), wounding of murine skin induces phosphorylation of Erk within a short period of time in all skin layers (FIG. 7 B). However, p-Erk was nearly absent in wounded or wound-proximal skin at 3, 7 or 12 days after wounding (FIG. 6 B, C), which is consistent with the findings on the declining p-Erk levels within hours from burn and excision injury in pigs (FIG. 6) and suggests that its induction is transient and diminishes shortly after wounding. Interestingly, the highest level of p-Erk was observed at day 12 in periepidermal fibroblasts (FIG. 6 B, C), which roughly corresponds to the time when fibroblasts are eliminated by immune cells or undergo apoptosis.

Next, we compared features of cells in homeostatic skin with those found inside the p-rpS6-zone and in the region immediately proximal to the zone. We found that cells positive for markers canonically related to the process of re-epithelialization and associated with epidermal thickening, such as an increase in keratinocyte size (FIG. 6 D) and increased keratinocyte proliferation (FIG. 6 E) are upregulated within the p-rpS6-zone. In respect to proliferation, we found that ~95% of keratinocytes positive for PCNA (a marker of proliferation) were also positive for p-rpS6 (FIG. 7 C). Moreover, we found that a commonly used marker of cellular senescence (p21CIP1/WAF1, Cdkn1a) was present nearly exclusively in keratinocytes positive for p-rpS6 (FIG. 6 F; FIG. 7 C). We confirmed the enrichment of the p-rpS6-zone in the markers of senescence by staining against DNA double strand breaks (DSBs) protein γ-H2A.X, which especially in combination with detection of telomeric sequences TelC, that enables measurements of irreparable DSBs at telomeres, telomere associated foci (TAF) and is a reliable marker of cellular senescence. Both, the total number of γ-H2A.X and TAF were highly increase in the keratinocytes of the p-rpS6-zone when compared to the keratinocytes immediately outside of the zone (FIG. 7 D). Similarly, the proto-oncogene c-Fos, which is involved in the mechanisms of healing-related differentiation and migration of keratinocytes was also found to be present almost exclusively in keratinocytes positive for p-rpS6 (FIG. 6 G; FIG. 7 C). Another marker of re-epithelialization, expression of the keratin 16 (K16) was present at 12 days after wounding almost solely exclusively inside the p-rpS6-zone (FIG. 6 H; FIG. 7 C). In contrast, CD3-positive T cells present in the dermal compartment of the wounded area were found to be predominantly negative for p-rpS6 (FIG. 7 C, E). Another essential marker of healing is angiogenesis, which can be characterized by an increase in density of blood vessels and appearance of endothelial tip cells—these express proteins such as Endocan (also known as Endothelial cell-specific molecule 1; Esm1). Consistently, we found that the dermal compartment of the p-rpS6-zone is enriched in blood vessels (FIG. 6 I), and is specifically enriched in endocan-positive tip cells, most of which are also p-rpS6 positive (FIG. 7 C, F).

In summary, our data from murine wounds show that the p-rpS6-zone is not only related to the immediate response to wounding, but is also present throughout the whole process of healing while being associated with re-epithelialization, induction of cellular senescence, c-Fos expression and angiogenesis.

Formation of the p-rpS6-Zone is Induced by DAMPs and Dependent on mTOR

The S235/236 phosphorylation site of rpS6, which we primarily focused on to define the p-rpS6-zone, can be phosphorylated by several proteins including S6K1 and 2, RSK1 and PKC linking it to several master regulators of cell physiology including mTOR and Erk.

To unravel the mechanism leading to the formation of the damage-induced p-rpS6-zone, we used a range of chemical compounds to manipulate key players in a variety of signaling pathways in an in vitro scratch assay using primary human dermal fibroblasts (HDFs). High levels of p-rpS6 are constitutively present in cell culture conditions due to the presence of foetal bovine serum (FBS) in cell culture media (FIG. 8 A). However, a short incubation period with basal media lacking FBS (basal media) reduced the overall level of p-rpS6 while preserving normal morphology of cells (FIG. 8 A). In these conditions, we observed that induction of a scratch results in a significant elevation of p-rpS6 in cells proximal to the damaged area forming a zone, reminiscent of that found in vivo (FIG. 9 A, B; FIG. 8 B). Consistent with experiments on live pigs (FIG. 3), induction of p-rpS6 occurs shortly after execution of the scratch and persists for a prolonged period of time (FIG. 9 A, B; FIG. 8 B). We further confirmed the scratch-dependent increase in p-rpS6 in the proximity to the insult in a model of keratinocytes, using HaCat, a spontaneously immortalized human keratinocyte cell line (FIG. 8 C).

In HDFs the scratch assay also induced an increase in p-Erk: while it occurred earlier than rpS6 activation, at 5 min after scratching, its levels declined over time and returned to the control level within 90 min (FIG. 9 C, D; FIG. 8 D). The assay thus sufficiently mirrors the early response to wounding in vivo and can be used to study pathways leading to the phosphorylation of rpS6.

Pre-treatment of cells with rapamycin, a potent inhibitor of mTOR, resulted in an almost complete abrogation of the p-rpS6 induction in the scratch assay, but did not affect Erk phosphorylation. In contrast, the use of U0126 (an inhibitor of MEK, the upstream regulator of Erk) had no significant effect on rpS6 phosphorylation (FIGS. 9 E, F; FIG. 8 E) while almost completely abrogating p-Erk-positive cells (FIGS. 9 G, H; FIG. 8 F). These results therefore suggest that the induction p-rpS6 and p-Erk are two independent events regulated separately by the upstream master regulators mTOR and MEK respectively.

Factors released immediately after wounding are grouped under the term damage-associated molecular patterns (DAMPs), and include proteins, lipids, nucleic acids and small compounds such as ATP or glutamate. While these normally reside inside cells and their organelles, they are released or passively leaked from damaged and dying cells. In order to establish whether DAMPs induce formation of the p-rpS6-zone in response to wounding, or if it is the wounded cells themselves which respond, we performed a “passive scratch assay”. Specifically, donor cells were lacerated using a needle (i.e. scratch assay was performed in the whole area of the culture vessel), releasing DAMPs into the cell culture media. This DAMP-containing media was then directly transferred to non-scratched recipient cells, which were then fixed after 30 min and stained. We found that the presence of DAMPs in culture media causes a striking and homogenous population of p-rpS6-positive recipient cells throughout the cell culture plate (FIG. 9 I, J).

To assess whether a functional mTOR complex is needed for the induction or maintenance of the p-rpS6-zone we either treated cells with rapamycin immediately prior to the addition of DAMPs (as in FIG. 9 E, F) or treated them with rapamycin later when the p-rpS6-zone was fully established (as in FIG. 9 I, J). For both modes of treatment cells were incubated with the drug for 30 min.

Surprisingly, we found that when rapamycin is added at the beginning of the assay, it prevents formation of the p-rpS6-zone when compared to the control (DI and RI; DMSO immediate and rapamycin immediate, respectively) while the same duration of time with the drug added after zone formation did not significantly affect the frequency of the p-rpS6-positive cells (DL and RL; DMSO late and rapamycin late, respectively) (FIG. 9 K, L).

Overall, these results show that DAMPs are causal to mTOR-mediated phosphorylation of rpS6 in cells proximal to the damage.

Loss of Circulation and Hypoxia Prevent Induction of the p-rpS6-Zone

Lack of circulation (ischemia) and the resulting lack of oxygen (hypoxia) is at the heart of many types of chronic wounds including venous, pressure and diabetic ulcers. It was also shown that hypoxia reduces the activity of mTOR. We therefore determined whether the hypoxic conditions resulting from a loss of circulation could affect the formation of the p-rpS6-zone resulting from skin injury.

Therefore, we performed burn and excision injuries on alive and sacrificed pigs—while burn injuries do not breach the skin surface they remain hypoxic when circulation is halted after death, while the tissue of excision injuries is exposed to the surrounding air and is never fully hypoxic. Three types of porcine skin biopsy samples were collected: (i) a set of injuries was performed on live anaesthetised animals and the samples were collected after 30 min (“alive”), (ii) a second set of injuries was performed simultaneously, the animals were sacrificed after 30 min and the samples were collected 30 min post-sacrifice (60 min post injury, “alive-dead”), (iii) a final set of injuries was performed 3 min after sacrifice and samples were collected after 30 min (“dead”; FIG. 10 A). The lack of circulation in the dead animal was clear macroscopically as the burn induced no erythema (FIG. 11 A), while skin surface temperature, which was measured prior, during and after burn changed only by 1-2 degrees (FIG. 11 B). Importantly, we noted large differences in the level of p-rpS6 induction (FIG. 10 B, C). In fact, while burn injuries from live animals showed a large quantity of p-rpS6 below the necrotic zone, the induction of the p-rpS6-zone was completely abrogated in burn samples collected from the recently-deceased animals (FIG. 10 B, C). Surprisingly, samples from animals that received burn injury while the animal was still alive, but were collected 30 min post-sacrifice showed no difference in the levels of p-rpS6 when compared to samples collected from alive animals (FIG. 10 B, C). In contrast to the sharp decline in p-rpS6 levels in the burn wounds of recently-deceased animals, the excision wounds did not show any significant differences in p-rpS6 levels in samples collected from alive, dead and “alive-dead” conditions (FIG. 10 D, E). We hypothesize that the difference in the response of the skin of the dead animals to the excision and burn wounds is due to the fact that an excision wound is exposed to atmospheric oxygen, allowing the regions nearest to the wound to activate the p-rpS6-zone, even when oxygen from the blood vessels is no longer circulating.

To verify this hypothesis, we tested the response of porcine skin ex vivo to the excision wound in conditions deprived of oxygen. To do this, skin was wounded in control (aerobic) conditions or in an anoxic tent in oxygen-deprived (anaerobic) conditions (FIG. 10 F). We observed that wounding skin in anaerobic conditions prevented the formation of the p-rpS6-zone (FIG. 10 G, H). Skin wounded under aerobic conditions formed a p-rpS6-zone comparable to that seen under in vivo conditions (FIG. 10 G, H). Interestingly, the zone around the ex vivo excision wound was consistently narrower than the zone seen in wounds performed in vivo, with the depth of the prior reaching approximately ~0.3 mm (FIG. 10 G), roughly matching the oxygen penetration depth in tissues.

Overall, these results show that circulation and oxygenation of tissue are needed for induction, but not maintenance of the p-rpS6-zone.

The p-rpS6-Zone Reports on the Progression of Healing and Delineates Highly Vascularized Regions in Pre-Clinical Studies

Adequate vasculature is the most important parameter when it comes to the clinical prognosis for the effectiveness of healing. As the p-rpS6-zone is functionally linked to tissue oxygenation we considered the possibility of using the zone to characterise healing states of wounds. To challenge the diagnostic value of an assessment of the formation of the p-rpS6-zone in a pre-clinical scenario, we characterized the properties of the zone in porcine skin samples collected 7 days post-wounding.

Pigs underwent liver surgery (an experiment unrelated to this study) that required an incision wound on the dorsal part of the abdomen. After the operation the wounds were stitched together, and 7 days later we observed that the effectiveness of healing varied greatly in between animals and even different sites of the same injury (FIG. 11 C). Trained veterinarians provided an unbiased healing efficiency score for each site of the wound from which samples were collected (FIG. 11 C).

In order to assess the relationship between vasculature and the p-rpS6-zone we quantified the number and size of (CD31-positive) blood vessels inside the zone and a size-matched region just outside of the zone (FIG. 10 I). We found that the zone consistently demonstrates a larger quantity of blood vessels, which are also on average larger in size (FIG. 10 I-K). It is worth stressing that this property of the zone is so consistent it allows distinguishing regions of poor and rich vasculature even within the same samples (FIG. 11 E).

We correlated the healing score with the area occupied by the p-rpS6-zone for each sample and found that the two are strongly inversely correlated: wounds with a larger p-rpS6-zone show a lower healing score (FIG. 10 L, M; FIG. 11 E). We also found that even in wounds that macroscopically appear healed, there is a small quantity of p-rpS6 left, that could be used to trace where the wound used to be (FIG. 12 F).

In summary, these results demonstrate that the p-rpS6-zone accurately reports on the status of the skin vasculature in preclinical samples and thus can be used to visualize the markers of healing as a predictor of healing outcome.

### Discussion:

The temporal dynamics of tissue response to injury and the healing process is relatively well understood with methods such as RT-PCR and WB providing essential information on the levels of factors driving inflammation, matrix remodelling and angiogenesis. However, the spatial characterization of these processes is still largely unknown with only recent research work suggesting how tissues of non-mammalian model organisms govern generation and propagation of signals necessary for healing. Multiple questions remain unanswered, such as how deep inside the tissue will cells undergo cell death and which will survive; where in the tissue does the response to wounding start and end, and what part of the damaged tissue initiates and drives its healing?

With the present invention, we show a phenotype of spatial response of skin to damage in mammals. The p-rpS6-zone defines an area of skin tissue starting at the end of the necrotic layer at the side of the skin injury. The zone forms within minutes after damage and continues throughout the process of healing. One of the features of the p-rpS6-zone is its stability and ease of detection. While other spatially-organized markers that originate from wounding such as ROS gradient or p-Erk waves operate on a basis of propagation of signal spikes or gradients that spread across the tissue, the p-rpS6-zone establishes itself as a stable zone of signal around the insult. This makes it easily detectable in all laboratories without usage of transgenic animals or in vivo reporter systems.

Despite the wide range of phenotypes characterizing response to wounding and the healing process, the one marker capable of defining a general response of skin to the presence of a wound is still missing. The p-rpS6-zone appears to comprise the core cellular processes related to healing, including cell growth, proliferation, senescence and angiogenesis. Although p-rpS6 has been seen in wounds before, it has never before been characterised as a stand-alone feature (Niethammer et al., Nature 459 (2009), 996-999; Tsai et al., Cells 11 (2022), 11050817). The definition of the p-rpS6-zone brings with it several advantages over classic histological wound size assessment and extrapolation of its spatial properties onto the surrounding skin architecture. For one, it is valuable to have an easily detectable single marker that represents multiple other features of healing and delineates the tissue which has responded to the initial damage. Also, it is likely that the p-rpS6-zone accurately reports on healing defects such as poor vasculature and hypoxia. Finally, the p-rpS6-zone is consistently activated by a wide range of wound types, including burns, excision, incision or needle pricks, which to our knowledge is unique. Thus, the characterization of the zone provides clinically relevant data that goes beyond the identification of the wounded region.

p-rpS6 can be Used to Visualize Damage/Wounding of Cartilage

For ex vivo experiments on cartilage, ears of a sheep were removed from the sacrificed animal and kept for acclimatization for 1 h in an incubator at 37 C. The tissue was then subjected to wounding using a 6 mm biopsy punch. At specific time points the edge of the original biopsy-wound was extracted and fixed in formalin. The samples were processed and stained against p-rpS6 as described in the sections above.

The results are disclosed in FIG. 13: p-rpS6 is induced upon damage of cartilage. (A) Sheep ear ex vivo was damaged by an excision wounding using a biopsy puncher. After 1.5 h samples were collected from the damaged and control (unwounded) sites. (B) The representative histological images show p-rpS6 staining of unwounded ear cartilage (top image) and the wounded site of the cartilage (bottom image). The scalebar is 100 μm.

Secondly, we investigated the hair follicles surrounding the wound (but not those that are distant from wounds) and found these to be positive for p-rpS6 (see below IHC for p-rpS6; all scale bars are 100 μm). The results are depicted in FIG. 14.

p-rpS6 can be Used to Visualize Hair Growth and Functionality of Hair Follicles

Hair follicles in the active phase are positive for p-rpS6.

For assessment of p-rpS6 signal in hair follicles we collected skin of active hair growth (anagen) visible by dark patches of skin pigmentation and compared it to the light skin (telogen) that is associated with the lack of hair growth. Histological sections were stained with IHC with antibodies against p-rpS6, Lef1 and pSTAT3 as described above (and well-known in the art, as e.g. in Ito et al., Nature 447 (2007), 316-320). Lef1 and p-STAT3 IHC stainings were compared side-by-side with p-rpS6 staining using sequential sections.

We collected dark and light patches of skin from five black-haired WT C57Bl/6 mice. The colour of these skin pieces corresponds to the growth phase of hair follicles (anagen=dark skin) and the lack of growth (telogen=light skin) respectively. We found that p-rpS6 was highly upregulated in the actively growing hair follicles (see the IHC for p-rpS6 below; all scale bars are 100 μm). The results are depicted in FIG. 15.

Moreover, we found an overlap between p-rpS6 and pathways/makers of hair follicle growth and regeneration: Lef1 and p-STAT3 (Y705). Using sequential histological sections, we found that anagen hair follicles of homeostatic skin are positive for both Lef1 and p-rpS6 (FIG. 16). Similarly, STAT3 signaling has been related to growth and function of hair follicles and the follicles proximal to the wound site are positive for both p-rpS6 and pSTAT3 Y705 (FIG. 17).

Based on this specification and the examples, the present invention therefore discloses the following preferred embodiments:
1. A method comprising the following steps:
   measuring an individual level or amount of presence, expression or activation of phosphorylated ribosomal protein S6 (p-rpS6) in a sample from a wound; and
   comparing the measured individual amount or level of p-rpS6 of the sample with a standard level or amount of p-rpS6 to assess tissue response to wounding;
or
a method comprising the following steps:
   measuring an individual level or amount of presence, expression or activation of phosphorylated ribosomal protein S6 (p-rpS6) in a sample from a wound; and
   determining whether the measured individual amount or level of p-rpS6 of the sample is indicative for an impaired state of wound healing;
or
a method comprising the following steps:
   measuring an individual level or amount of presence, expression or activation of phosphorylated ribosomal protein S6 (p-rpS6) in a sample from a wound of a patient;
   comparing the measured individual amount or level of p-rpS6 of the sample with a standard level or amount of p-rpS6 to assess tissue response to wounding; and
   in case the comparison results in an assessment of impaired tissue response to wounding administering an effective amount of a therapeutic agent for improving wound healing in the patient.
2. A method comprising the following steps:
   measuring the presence or absence of phosphorylated ribosomal protein S6 (p-rpS6) in a sample from a wound of a patient; and
   identifying the absence of p-rpS6 in the sample as being indicative for a lack of vascularisation and/or lack of oxygenation of the wound and/or presence of necrotic tissue within the wound.
3: A method for identifying necrotic tissue within a wound comprising the following steps:
   measuring the presence or absence of phosphorylated ribosomal protein S6 (p-rpS6) in a sample from a wound; and
   identifying the area from which the sample of the wound was derived as being necrotic tissue; and, if applicable,
   removing the necrotic tissue from the wound
4. A method comprising the following steps:
   measuring an individual level or amount of presence, expression or activation of phosphorylated ribosomal protein S6 (p-rpS6) in a sample from a wound; and
   diagnosing an impaired wound healing risk if the measured individual amount or level of p-rpS6 of the sample is increased in comparison with the amount or level of a wound sample of a patient which is not affected by an impaired wound healing risk.
5. Method according to any one of embodiments 1 to 4, wherein an increased level or amount of presence, expression or activation of p-rpS6 measured compared to the wound of an individual with no risk for an impaired wound healing is indicative for a risk for developing a chronic wound, including venous, pressure and diabetic ulcer; a hypertrophic scarring, a hypoxia-injured tissue, an impaired re-epithelialization, or an increased cell death in said tissue.
6. Method according to any one of embodiments 1 to 5, wherein the level or amount of presence, expression or activation of p-rpS6 measured in a wound of the skin provides an estimation of the depth of skin affected by cell death and/or an identification of the tissue regions that are affected by wounding but not undergoing an immediate death.
7. Method according to any one of embodiments 1 to 6, wherein the method is a method of determining a diagnosis of the wound in a subject.
8. Method according to any one of embodiments 1 to 7, wherein the measurement is repeated for at least 1, 2, 3, 4, or 5 times.
9. Method according to any one of embodiments 1 to 8, wherein the measurement is repeated during a course of monitoring the wound.
10. Method according to any one of embodiments 1 to 9, wherein the determination or comparison is implemented and/or performed at least in part by a computer, by a computer processor and/or by a computer program.
11. Method according to any one of embodiments 1 to 10, wherein the method comprises the step of evaluating the wound visually.
12. Method according to any one of embodiments 1 to 11, wherein the method comprises communicating a result via a communication medium, preferably wherein the communication medium is a computer file, an email, a fax, or a paper document.
13. Method according to any one of embodiments 1 to 12, wherein the sample is a tissue sample from the wound or a cartilage sample from the wound or from the vicinity of the wound, preferably wherein the sample is from a marginal edge of the wound, especially wherein the sample comprises keratinocytes or other type of skin cells isolated or purified from the wound.
14. Method according to any one of embodiments 1 to 13, wherein the measured levels are normalized fluorescence intensity (NFI) per mg protein, especially wherein the measured values are normalized expression values.
15. Method according to any one of embodiments 1 to 14, wherein the wound is a burn injury, an excision injury, a puncture injury, a diabetic ulcer, a pressure ulcer, a venous stasis ulcer, a radiation ulcer, a skin injury, an unhealed surgical wound, a wound from a surgical procedure, a wound from a peripheral vascular disease, a wound from a complication of trauma, a wound in a cancer patient, a wound in a patient receiving a steroid therapy, a wound from an inflammatory skin disease, a chronically impaired cutaneous wound, or any combination thereof, especially wherein the wound is a burn injury, an excision injury, or a puncture injury.
16. Method according to any one of embodiments 1 to 15, wherein the sample from a wound is a sample taken as a biopsy from the wound or its vicinity until approximately about 2 mm from the wound, a blood sample taken from the wound, a blood sample of the patient having the wound, or a body fluid sample and/or tissue sample and/or cell sample from the circulation of the patient having the wound.
17. A method of treating an individual having a wound with an individual level or amount of presence, expression or activation of p-rpS6 in a sample from said wound indicating an impaired tissue response to wounding by administering an effective amount of a therapeutic agent for improving wound healing in the patient.
18. Method according to embodiment 17, wherein the treating comprises administering to the individual a pharmaceutical composition that comprises an inhibitor of POKinase signaling, preferably selected from an inhibitor of POKinase complex, Akt, mTOR, 4E.BP1, Ribosomal Protein S6 Kinase, HIF-Iα, PTEN, POKinase, IGFRPB3, Src, GSK3, β-catenin, any protein that is a member of the insulin signaling or POKinase signaling pathways of proteins; an activator of the thrombin receptor, preferably thrombin or a thrombin receptor activating peptide (TRAP), such as TRAP6, TRAP7 or TRAP8; an activator of insulin signaling through IGF-1R, preferably selected from the group consisting of insulin, insulin-like growth factor 1 (IGF-1), IGF-2, PTEN, POKinase, mTOR, 4E.BP1, Ribosomal Protein S6 Kinase, HIF-1a, PTEN, IGFRPB3, Src, GSK3, β-catenin, any protein that is a member of the insulin signaling or POKinase signaling pathways of proteins; or any combination thereof.
19. A kit, preferably for use in the method according to any one of embodiments 1 to 16, comprising
   means for taking a wound sample from a patient; and
   means for detecting the level or amount of presence, expression or activation of p-rpS6. 20. Kit according to embodiment 19, wherein the means for detecting the level or amount of presence, expression or activation of p-rpS6 are selected from labelled specific p-rpS6-binding molecules, preferably fluorescence-labelled anti-p-rpS6-antibodies, especially fluorescence-labelled monoclonal anti-p-rpS6-antibodies.
21. The kit of any one of embodiments 19 or 20, wherein the kit further complies an instruction manual.
22. Method for visualizing hair growth or functionality of hair follicles in a sample comprising hair follicles, comprising the following steps:
   measuring an individual level or amount of presence, expression or activation of phosphorylated ribosomal protein S6 (p-rpS6) in a sample comprising hair follicles; and
   comparing the measured individual amount or level of p-rpS6 of the sample with a standard level of healthy hair follicles or amount of p-rpS6 to assess the functionality of the hair follicle;
or
a method comprising the following steps:
   measuring an individual level or amount of presence, expression or activation of phosphorylated ribosomal protein S6 (p-rpS6) in a sample comprising hair follicles; and
   determining whether the measured individual amount or level of p-rpS6 of the sample is indicative for an impaired state of the hair follicle;
or
a method comprising the following steps:
   measuring an individual level or amount of presence, expression or activation of phosphorylated ribosomal protein S6 (p-rpS6) in a sample comprising hair follicles of a patient;
   comparing the measured individual amount or level of p-rpS6 of the sample with a standard level or amount of p-rpS6 to assess healthy hair growth; and
   in case the comparison results in an assessment of impaired hair follicle function administering an effective amount of a therapeutic agent for improving hair growth in the patient.
23. A method comprising the following steps:
   measuring the presence or absence of phosphorylated ribosomal protein S6 (p-rpS6) in a sample comprising hair follicles of a patient; and
   identifying the absence of p-rpS6 in the sample as being indicative for an impaired hair growth.
24. A method comprising the following steps:
   measuring an individual level or amount of presence, expression or activation of phosphorylated ribosomal protein S6 (p-rpS6) in a sample comprising hair follicles; and
   diagnosing an impaired hair follicle activity if the measured individual amount or level of p-rpS6 of the sample is increased in comparison with the amount or level of a sample of a patient which is not affected by an impaired hair growth.
25. Method according to any one of embodiments 22 to 24, wherein the hair follicles are also tested with respect to Lef1 and/or pSTAT3 Y705, wherein presence of Lef1 and/or p-STAT3 Y705 indicates appropriate function of the hair follicles and healthy hair growth.

1. A method comprising the following steps:
measuring the presence or absence of phosphorylated ribosomal protein S6 (p-rpS6) in a sample from a wound of a patient; and
identifying the absence of p-rpS6 in the sample as being indicative for a lack of vascularisation and/or lack of oxygenation of the wound and/or presence of necrotic tissue within the wound.

2. A method for identifying necrotic tissue within a wound comprising the following steps:
measuring the presence or absence of phosphorylated ribosomal protein S6 (p-rpS6) in a sample from a wound; and
identifying the area from which the sample of the wound was derived as being necrotic tissue; and, if applicable,
removing the necrotic tissue from the wound.

3. A method comprising the following steps:
measuring an individual level or amount of presence, expression or activation of phosphorylated ribosomal protein S6 (p-rpS6) in a sample from a wound of a patient;
comparing the measured individual amount or level of p-rpS6 of the sample with a standard level or amount of p-rpS6 to assess tissue response to wounding; and
in case the comparison results in an assessment of impaired tissue response to wounding administering an effective amount of a therapeutic agent for improving wound healing in the patient.

4. A method comprising the following steps:
measuring an individual level or amount of presence, expression or activation of phosphorylated ribosomal protein S6 (p-rpS6) in a sample from a wound; and
diagnosing an impaired wound healing risk if the measured individual amount or level of p-rpS6 of the sample is increased in comparison with the amount or level of a wound sample of a patient which is not affected by an impaired wound healing risk.

5. Method according to claim 1, wherein an increased level or amount of presence, expression or activation of p-rpS6 measured compared to the wound of an individual with no risk for an impaired wound healing is indicative for a risk for developing a chronic wound, including venous, pressure and diabetic ulcer; a hypertrophic scarring, a hypoxia-injured tissue, an impaired re-epithelialization, or an increased cell death in said tissue.

6. Method according to claim 1, wherein the level or amount of presence, expression or activation of p-rpS6 measured in a wound of the skin provides an estimation of the depth of skin affected by cell death and/or an identification of the tissue regions that are affected by wounding but not undergoing an immediate death.

7. Method according to claim 1, wherein the method is a method of determining a diagnosis of the wound in a subject, preferably wherein the measurement is repeated during a course of monitoring the wound; and/or wherein the measurement is repeated for at least 1, 2, 3, 4, or 5 times.

8. Method according to claim 1, wherein the determination or comparison is implemented and/or performed at least in part by a computer, by a computer processor and/or by a computer program.

9. Method according to claim 1, wherein the method comprises the step of evaluating the wound visually.

10. Method according to claim 1, wherein the method comprises communicating a result via a communication medium, preferably wherein the communication medium is a computer file, an email, a fax, or a paper document.

11. Method according to claim 1, wherein the sample is a tissue sample from the wound, preferably wherein the sample is from a marginal edge of the wound, especially wherein the sample comprises a keratinocyte isolated or purified from the wound.

12. Method according to claim 1, wherein the measured levels are normalized fluorescence intensity (NFI) per mg protein, especially wherein the measured values are normalized expression values.

13. Method according to claim 1, wherein the wound is a burn injury, an excision injury, a puncture injury, a diabetic ulcer, a pressure ulcer, a venous stasis ulcer, a radiation ulcer, a skin injury, an unhealed surgical wound, a wound from a surgical procedure, a wound from a peripheral vascular disease, a wound from a complication of trauma, a wound in a cancer patient, a wound in a patient receiving a steroid therapy, a wound from an inflammatory skin disease, a chronically impaired cutaneous wound, or any combination thereof, especially wherein the wound is a burn injury, an excision injury, or a puncture injury.

14. A kit for use in the method according to claim 1, wherein the kit comprises
means for taking a wound sample from a patient; and
means for detecting the level or amount of presence, expression or activation of p-rpS6.

15. The kit according to claim 14, wherein the means for detecting the level or amount of presence, expression or activation of p-rpS6 are selected from labelled specific p-rpS6-binding molecules, preferably fluorescence-labelled anti-p-rpS6-antibodies, especially fluorescence-labelled monoclonal anti-p-rpS6-antibodies; and/or wherein the kit further complies an instruction manual.
